(19) United States
(12) Patent Application Publication
(10) Pub. No.: US 2015/0252425 A1
(43) Pub. Date: Sep. 10, 2015

(54) GENE EXPRESSION PROFILING FOR THE DIAGNOSIS OF PROSTATE CANCER

(71) Applicant: Caldera Health Ltd., Auckland (NZ)

(72) Inventors: James Douglas WATSON, Auckland (NZ); Clare ELTON, Auckland (NZ); David Rex MUSGRAVE, Hamilton (NZ); Helene BELANGER, Auckland (NZ); Kay Alison APPLEYARD, Hamilton (NZ); Kristen CHALMET, Auckland (NZ)

(21) Appl. No.: 14/311,136

(22) Filed: Jun. 20, 2014

Related U.S. Application Data
(60) Provisional application No. 61/948,486, filed on Mar. 5, 2014.

(51) Int. Cl. C12Q 1/68

(52) U.S. Cl. CPC C12Q 1/6886; C12Q 2600/16; C12Q 2600/158; C12Q 2600/112

(57) ABSTRACT

Methods for diagnosing the presence of a disorder, such as prostate cancer, in a subject are provided, such methods including detecting the relative frequency of expression of RNA biomarkers in a biological sample obtained from the subject, for example, using NGS technology and comparing the relative levels of expression with predetermined threshold levels. Levels of expression of at least two of the RNA biomarkers that are above or below the predetermined threshold levels are indicative of the presence of prostate cancer in the subject. Also provided is a method for preparing a reference standard for quantitating the relative frequency of expression of RNA biomarkers in a biological sample obtained from the subject with a prostate cancer lesion using NGS technology.

### REFERENCE TO RELATED APPLICATIONS

This application claims priority to U.S. provisional patent application No. 61/948,486, filed Mar. 5, 2014.

### SEQUENCE LISTING

The instant application contains a Sequence Listing which has been submitted electronically in ASCII format and is hereby incorporated by reference in its entirety. Said ASCII copy, created on Jun. 11, 2014, is named 24700.1010NP_SL.txt and is 729 KB in size.

### TECHNICAL FIELD

The present disclosure relates to methods using next generation sequencing and RNA biomarker compositions for diagnosing and defining the staging or progress of disorders such as prostate cancer.

### BACKGROUND

The use of prostate specific antigen (PSA) as a diagnostic biomarker for prostate cancer was approved by the US Federal Drug Agency in 1994. Together with a digital rectal examination (DRE) and prostate biopsy to determine a Gleason score to stage the cancer, the PSA test has remained the primary test for use in prostate cancer diagnosis and in monitoring disease recurrence. In May 2012, the United States Preventive Services Task Force (USPSTF) downgraded its recommendation on using the PSA test for prostate cancer screening to a “D” (Annals of Internal Medicine, May 22, 2012). The USPSTF adopted this position because it decided “there is moderate or high certainty that the service has no net benefit or that the harms outweigh the benefits.”

A blood serum level of around 4 ng per ml of PSA is considered indicative of prostate cancer, while a PSA level of 10 ng per ml or higher is considered highly suggestive of prostate cancer. If the results of the PSA test and the DRE are abnormal, a biopsy is generally performed in which small samples of tissue are removed from the prostate and examined. A Gleason score based on cellular changes in the prostate has predictive value in the range of Gleason 2-4 and Gleason 8-10, that is, at either end of the Gleason spectrum. The predictive value for men who present with a Gleason score of 5-7 is more uncertain and this latter range is where the majority of men present. In particular, a Gleason score of 6 encompasses men who may have an indolent form of disease, and also men who are at high risk for cancer progression.

As a result many men diagnosed with cancer with a Gleason score of 6 and having an indolent cancer are treated unnecessarily at high cost to health systems as well as risking the patient's quality of life, such as through incontinence or impotence. Prostate cancer is the most prevalent form of cancer and the second most common cause of cancer death in New Zealand, Australian and North American males (Jemal et al. CA Cancer J. Clin., 57(1):43-66, 2007).

The use of the PSA test cannot simply be dropped until a new test is available because at least some of the men incubating life threatening forms of prostate cancer presenting with rising PSA levels would be missed until their cancers are well advanced and treatment options are more limited. Due to the economic costs of national screening, the need to avoid unnecessary over-treatment, the loss of quality of life, and/or the presence of progressive cancers producing only low or background levels of PSA, the need for a better diagnostic test for prostate cancer could not be clearer.

The need for gene-based tests is also evident as histologically identical prostate cancers can develop quite different clinical behaviors. In some men diagnosed with prostate cancer, the disease progresses slowly while in other men the disease progression can be rapid. There is a need today for a greater understanding of the genetic changes responsible for these behaviors in prostate cancer to enable better management of the cancer in patients. New tests are required not only for more accurate primary diagnosis, but also for assessing the risk of spread of primary prostate cancers, and for monitoring responses to therapeutic interventions.

There are further reasons underlying the need for gene-based diagnostics of this type for prostate cancer. Prostate adenocarcinomas, that is cancer of epithelial cells in the prostate gland, account for approximately 95% of prostate cancers, while the neuroendocrine cancers are rare but account for some 5% of prostate cancers,

Some 15 to 17% of men with prostate adenocarcinomas have cancers that progress without producing high- or increasing blood levels of PSA. In these patients, who are termed asymptomatic, the PSA test often returns false negative test results as the cancer grows without significant PSA changes.

Benign prostate hypertrophy (BPH), a non-malignant growth of epithelial cells, and prostatitis, caused by an infection of the prostate gland, are diseases of the prostate that are often accompanied by increases in PSA levels, yielding false positives in the PSA test. Both BPH and prostatitis are common in men over 50, with a prevalence rate of 2.7% for men aged 45-49, increasing to at least 24% by the age of 80 years (Ziada et al. (1999) Urology 53(3 Suppl 3D):1-6). Bacterial infection of the prostate can be demonstrated in only about 10% of men with symptoms of chronic prostatitis/chronic pelvic pain syndrome. Bacteria able to be cultured from patients suffering chronic bacterial prostatitis are mainly Gram-negative uropathogens.

Another condition, known as prostate intraepithelial neoplasia (PIN), may precede prostate cancer by five to ten years. Currently there are no specific diagnostic tests for PIN, although the ability to detect and monitor this potentially pre-cancerous condition would contribute to early detection and enhanced survival rates for prostate cancer.

### Gene Expression Profiling

Gene expression is the transcription of DNA (deoxyribonucleic acid) into messenger RNA (messenger ribonucleic acid; also referred to as gene transcripts) by RNA polymerase. Up-regulation describes a gene which has been observed to have higher expression (higher RNA levels) in one sample (for example, from cancer tissue) compared to another sample (usually healthy tissue from a control sample). Down-regulation describes a gene which has been observed to have lower expression (lower RNA levels) in one sample (for example, from cancer tissue) compared to another sample (usually healthy tissue from a control sample).

Differential changes in gene expression underlie the phenotype of prostate cancer, which varies from one patient to another. Such gene expression changes involve cellular pathways that variously affect cellular and tissue morphologies, growth rates, cellular adhesion, responsiveness to androgens and pharmacological blocking agents for androgens, and varying metastatic potential. Thus prostate cancer progression involves multiple steps, and may result in progression from a localized indolent cancer state to invasive carcinoma and metastasis. The progression of prostate cancer likely proceeds, as seen for other cancers, via events that include the loss of function of cell regulators such as cancer suppressors, cell cycle and apoptosis regulators, proteins involved in metabolism and stress response, and metastasis related molecules (Abate-Shen et al. Polypeptides Dev. 14(19):2410-34, 2000; Ciocca et al. Cell Stress Chaperones 10(2):86-103, 2005).

While specific patterns of gene expression have been reported for prostate cancer and a number of candidate genes and pathways likely to be important in individual cases have been identified, these have not met with consistent agreement from study to study (Tomlins et al., Annu. Rev. Pathol. 1:243-71, 2006). Such studies generally involve comparing gene expression patterns of primary carcinomas to adjacent gland tissue used as the normal prostate control tissue, or retrospective analysis of prostate carcinoma tissue samples of known clinical outcome and selecting signature genes that correlate with Gleason scores and survival or mortality to derive a prostate cancer gene expression (GEX) score for predicting the probability of relapse of cancer in individuals (for example; Lapointe et al., Proc. Natl. Acd. Sci. USA; 101, 811-816: 2004; Chudin et al., U.S. Pat. No. 7,914,988).

Again, there is little agreement or consistency between gene transcript candidates from different studies and one result is that diagnostics for prostate cancer based on the gene-based biomarker candidates have yet to have an impact clinically.

It is likely that one major cause of the inconsistent results from study to study arise from the influence of field effects on the fold change estimates of gene expression when comparing tumor cell RNA levels to adjacent tissues used as healthy control tissues.

Another reason for inconsistent results appear to arise from processing differences; in the integrity of the tissue samples used as well as the biological heterogeneity of prostate cancers, in platform technologies such as cDNA microarray and RT PCR, and in analytical tools. Formalin fixed paraffin embedded (FFPE) tissues allow a convenient comparison of tumor and adjacent tissues in retrospective studies while many of the cDNA microarray studies have used snap frozen tissues (Bibikova et al., Genomics 89:666-72, 2007; van't Veer et al., Nature 415:530-6, 2002). In addition, some studies have included accident victim donors as controls to overcome potential field effects (Aryee et al. Sci Transl Med 5, 169ra10 2013; Chandran et al. BMC Cancer, 5:45 doi: 10.1186/1471-2407-5-45, 2005).

Studies have involved hundreds of candidate genes that at the end of such processes yield few that share only a moderate consensus. However, there are a few genes that have been shown to have probable roles in prostate carcinogenesis, including hepsin (HPN; Rhodes et al., Cancer Res. 62:4427-33, 2002), alpha-methylacyl-CoA racemase (AMACR; Rubin et al., JAMA 287:1662-70, 2002; Lin Biosensors 2:377-387, 2012), enhancer of Zeste homolog 2 (EZH2; Varambally et al. Nature, 419:624-9, 2002), L-dopa decarboxylase (DDC; Koutalellis et al. BJU International, 110:E267-E273, 2012), and anterior-gradient 2 (AGR2; Hu et al. Carcinogenesis 33:1178-1186, 2012).

More recently, bioinformatic approaches employing data from gene expression profiling using microarray technology have generated lists of dysregulated genes in prostate cancer. These studies have also shown few consensus genes (Aryee et al. Sci Transl Med 5, 169ra10, 2013; Chandran et al. BMC Cancer, 5:1471-2407 2005; Pflueger et al. Genome Res. 21:56-67, 2011; Prensner et al. Nature Biotechnology 29:742-749, 2011; Shancheng Ren et al. Cell Research 22:806-821, 2012; Glinsky et al., J. Clin. Invest. 113:913-23, 2004; Hsieh et al, Nature doi:10.1038/Nature.10912, 2012; Lapointe et al, Proc. Natl. Acad. Sci. USA 101:811-6, 2004; LaTulippe et al, Cancer Res. 62:4499-506, 2002; Markert et al, Proc. Natl. Acad. Sci. Doi:10.1073/pnas.1117029108, 2012; Rhodes et al, Cancer Res. 62:4427-33, 2002; Singh et al, Cancer Cell 1:203-9, 2002; Yu et al, J. Clin. Oncol. 22:2790-9, 2004; Varambally et al., Nature 419:624-9, 2002).

### Androgens and Prostate Cancer

Androgens such as dihydrotestosterone (dHT) and testosterone are the key drivers of prostate cancer. Gene transcription changes that initiate carcinogenesis must arise from the binding of DHT (and testosterone) to the androgen receptor (AR) but have not been exploited widely in prostate cancer gene expression profiling. The AR is a transcription factor and is a member of the nuclear receptor superfamily. The transformation to prostate cancer has been linked to several somatic AR gene mutations and changes in AR protein complex formation, which in turn increase the potential activity of the AR (Wilson Reproduction, fertility, and development, 13:673-8, 2001; Heinlein et al., Endocrine reviews, 25: 276-308, 2004). The AR with co-regulators induces expression of target genes, such as prostate specific antigen (Kallikrein 3) and Kallikrein-related peptidase 2 in prostate (Kim et al., Journal of Cellular Biochemistry, 93:233-41, 2004). The AR activity is also regulated by growth factor cascades which can induce AR modifications, including phosphorylation and acetylation or changes in interactions of the AR with other cofactors. Epidermal growth factor (EGF), Insulin-like growth factor 1 (IGF-1), Interleukin-6 and ligands stimulating the protein kinase A pathways activate the AR by phosphorylation in the absence of androgens either directly or indirectly via the mitogen-activated protein kinase (MAPK) cascade and induce AR gene expression (Culig Growth Factors, 3:179-84, 2004). These growth factors likely also play a role in field effects.

Androgens also induce rapid activation of kinase-signaling cascades and modulate intracellular calcium levels. These effects are non-genomic as they occur in cells in the presence of inhibitors of transcription and translation, and occur too rapidly to involve transcription (Heinlein et al., Molecular Endocrinology, 16:2181-7, 2002; Lange et al., Annual Review of Physiology: 69:171-199, 2007).

In response to dHT, the AR interacts with the SH3 domain of tyrosine kinase v-src and viral oncogene homolog (c-src) (Migliaccio et al., EMBO Journal, 19(20):5406-17, 2000) to stimulate the mitogen-activated protein kinase (MAPK) signaling cascade and mitogen-activated protein kinase 1 (MAPK1). The AR can also activate the phosphoinositide-3-kinase (PI3K)/AKT kinase pathway in response to natural androgen. Such androgenic activation of PI3K leads to phosphorylation of AKT (Migliaccio et al., EMBO J 16; 19(20):5406-17, 2000; Sun et al., J. Biological Chemistry; 278(44):42992-3000, 2003; Castoria et al., Steroids; 69(8-9):517-22, 2004).

The interaction between phosphatase and tensin homolog (PTEN) and the AR inhibits the nuclear translocation of AR and promotes its degradation, which results in suppression of AR transactivation and induction of apoptosis (Lin et al., Molecular endocrinology; 18(10):2409-23, 2004; Mulholland et al., Oncogene; 25(3):329-37, 2006).

In contrast to gene transcript studies, genomic approaches to prostate cancer diagnosis and monitoring have involved DNA sequence changes and methylation, gene fusions, RNA splice variants and RNA editing. Genomic changes identified include the fusion of androgen-regulated genes, including transmembrane protease, serine 2 (TMPRSS2) with members of the erythroblast transformation specific (ETS) DNA transcription factor family (Tomlins et al., Science 310:644-8, 2005, Tomlins, Nature 448: 595-599, 2007). These fusions appear commonly in prostate cancers and have been shown to be prevalent in more aggressive cancers (Attard et al., Oncogene 27:253-63, 2008; Barwick et al. Br. J. Cancer 102:570-576, 2010; Demichelis et al., Oncogene 26:4596-9, 2007; Nam et al., Br. J. Cancer 97:1690-5, 2007). Transcriptional modulation of TMPRSS2-ERG fusions has been shown to be associated with prostate cancer biomarkers and TGF-beta signaling (Brase et al., BMC Cancer 11:507 doi: 10.1186/1471, 2011). In addition to specific gene fusions, a large number of mutational changes, including copy number variants, have been associated with prostate cancer tumors (Berger et al., Nature 470:214-220, 2011; Demichellis et al., Proc. Natl. Acad. Sci. doi:10.1073/pnas.117405109, 2012; Kumar et al., Proc. Natl. Acad. Sci. 108:17087-17092, 2011). Intratumor heterogeneity has also been found which has been suggested to result in underestimation of the degree of tumor heterogeneity (Gerlinger et al., New Eng, J. Med. 66:883-892, 2012). In particular mutations involving the substrate binding cleft of SPOP, which was found in 6-15% of prostate tumors, lacked ETS family gene rearrangements, suggesting that tumors with SPOP mutations define a new class of prostate tumors. Also tumors with SPOP mutations lacked PTEN deletions in primary tumors but not in metastatic tumors (Barbieri et al., Nature Gen. 44:685-689, 2012).

### Field Effects

In cancer, field effects refer to the occurrence of genetic and biochemical changes in structurally intact cells in histologically normal tissues adjacent to cancerous lesions. First described in the 1950s as field “cancerization”, this was originally based on morphological changes in cells surrounding cancerous lesions but with progress in molecular biology this description of field effects has changed from a histological to a more molecular definition.

In prostate cancer there are reported rates of up to 90% of prostate glands containing two or more cancerous foci at the time of clinical diagnosis (Andreoiu et al., Human Pathology, 41, no. 6, pp. 781-793, 2010). These multifocal lesions complicate staging of a cancer because individual foci usually display heterogeneity. It has been argued that the Gleason score, which combines the first most common with the second most common grade of dedifferentiation of cells, enhances the accuracy of prognosis (Iczkowski et al., Current Urology Reports, 12, no. 3, pp. 216-222, 2011) but does not account for the causes of the development of multifocality. Different cancerous foci could evolve independently from each other, remain isolated or fuse if they are in close proximity to each other. Alternatively, migratory cells, such as monocytes or lymphocytes attracted by developing cancerous cells, may produce cytokines or other mediators that cause gene expression changes in both cancerous and normal prostate epithelial cells.

The inflammation associated with the pathogenesis of prostate cancer may result from increased activity of inflammatory cytokines, particularly IL-6. For example, peripheral blood mononuclear cells interacting with cancerous prostate cells increase production of pro-inflammatory cytokines (Salman et al., Biomedicine & Pharmacotherapy 66(5):330-333, 2012). Inflammatory cytokines are likely candidates for driving field effects as they are secreted from cells, diffuse widely and rapidly, and some activate the androgen receptor.

Gene expression profiles which have been associated with aggressive clinical recurrence of disease after treatment show a clear association with the gene-expression profiles in adjacent normal-appearing tissue (Klein et al., J. Clin. Oncol. 31, 2013 suppl; abstr 5029).

### Adjacent Glandular Tissues

In many prostate cancer studies of differential gene transcripts, analyses depend on adjacent glandular tissues as “normal” or healthy tissue. However, this practice has certain limitations.

First, a selection of ‘normal’ glandular tissue based on morphology does not take into account field effects that change levels of expression occurring in these cells that are not visible in their morphology. Most of the tumors that develop in the prostate gland develop from the secretory epithelial cells located next to the lumen of the gland, which enables the rapid spread of molecules secreted by the tumor or migratory immune cells. As tumorigenesis progresses as indicated by the Gleason score, the field effect likely increases because the physical distance to normal tissue will decrease and secretions will reach the ‘normal’ areas more rapidly.

Second, as tumorigenesis progresses, the cancerous tissue will take up more of the gland and will start spreading, and it will become increasingly difficult to isolate a part of the gland that contains no cancerous tissue. As chances increase to include cancer cells in the ‘normal’ section with increasing Gleason scores, the adjacent glandular samples become less suitable to act as a representative of ‘normal tissue’.

### From Single to Multiple Biomarkers

The basic problem with the PSA test is that it is a one blood protein biomarker test which fails to detect some prostate cancer and is not prognostic, unable to reflect the disease heterogeneity. A single biomarker does not allow tumors of different lethality or aggressiveness to be differentiated so it offers little in terms of selecting treatment options.

Multiple gene expression biomarkers offer both diagnostic and prognostic value in one test. They reflect multiple gene changes as transcripts and overcome the problem of trying to use genomic or DNA tests such as those for methylation, mutations, deletions and gene fusions alone as biomarkers, which are limited because DNA tests do not reflect usage in cells.

An altered genome may contain variant point mutations, translocations, fusions and other changes but they might not reside in coding regions of the genome.

Microarray and RT-qPCR are commonly used as technologies to quantitate gene expression profiles in cancer and healthy tissue samples. Each has drawbacks such as time involved and costs in comparing gene expression levels across different patient samples, as well as requiring complicated normalization methods that may not be suitable for integration into a diagnostic test. Very often these transcripts, for which differential expression is difficult to measure, are the ones with the most diagnostic and/or prognostic value. RT-qPCR only allows limited multiplexing, which causes a rise in cost per RNA biomarker and hence in the overall cost of the diagnostic test.

A key advance in DNA sequencing is next generation sequencing technologies where billions of independent DNA sequence reads are generated in parallel, with each read derived from a single molecule of DNA. Next Generation Sequencing (NGS) will likely fill a diagnostic need in the field of inheritable disorders where genomic changes are sought, but to date it has not been applied to the diagnostic needs in cancer. There are no commercial tests to date that employ NGS in multiple RNA biomarker expression profiling as a platform for cancer diagnostics and cancer staging.

The present invention addresses the need for a more accurate prostate cancer primary diagnosis, a better assessment of the risk of spread of primary prostate cancers and the need for new tools for monitoring responses to therapeutic interventions.

### SUMMARY

The present invention provides methods for determining the presence and progression of a disorder, such as a cancer, for example prostate cancer, in a subject. Such methods involve the clinical application of gene transcript changes as biomarkers for diagnosing disorders such as prostate cancer, together with the use of next generation sequencing (NGS) advances to perform diagnostic tests. The methods and compositions disclosed herein are employed in combination to determine the relative frequency of expression of one or more RNA biomarkers (also referred to as gene transcript biomarkers) specific for the disorder in the tested subject compared to that in healthy controls. Disorders that can be diagnosed and monitored using the methods disclosed herein include, but are not limited to, cancers, such as prostate and breast cancers.

Determination of the relative frequency of expression of specific combinations of RNA biomarkers using the methods disclosed herein can also be used to determine the type and/or stage of a disorder, and to monitor the progression of a disorder and/or the effectiveness of treatment. In certain embodiments, the disclosed methods determine changes in frequency of expression of RNA biomarkers in order to distinguish between indolent, or insignificant, forms of a cancer (such as prostate cancer), which have a low likelihood of progressing to a lethal disease, and aggressive, or significant, forms of cancer which are life threatening and require treatment. (For a discussion of significant versus insignificant forms of prostate cancer, see Ploussard et al. European Urology 60:291-303, 2011). The disclosed methods can thus be employed to identify subjects at risk of developing metastatic cancer and/or having an increased risk of cancer recurrence. Subjects identified as having aggressive cancer, or being at increased risk of developing metastatic cancer, can be treated using known therapeutic regimens. Such individuals may, or may not, exhibit any of the traditional risk factors for metastatic disease.

The methods disclosed herein allow the determination of the relative frequency of expression of multiple RNA biomarkers simultaneously. Oligonucleotides specific for multiple biomarkers are amplified individually at the same time to produce a pool of amplicons and a multiplex format is then used to identify and quantify all the amplicons simultaneously using next generation sequencing (NGS). The advantages of this simultaneous sequencing are a reduction in cost and the amount of tissue required, an increase in the level of reproducibility due to less hands-on manipulation, and increased throughput.

More specifically, the disclosed methods employ oligonucleotides specific for RNA biomarkers identified as being associated with the presence and/or progression of a disorder, such as prostate cancer, at specific steps of a NGS protocol to selectively quantitate cDNAs complementary to the RNA biomarkers and compare their relative frequency of expression between a test subject and healthy donors, thereby determining the presence or absence of the disorder in the test subject as well as defining differences in expression between different stages of the disorder.

In conventional NGS methodologies for RNA expression analysis (also referred to as RNA-seq), the actual frequency of expression of each transcript is determined for the whole genome. These frequencies can be biased by differences in the efficiency of the cDNA production, large variations in abundance and size of the transcript, subsequent PCR amplification steps for each transcript, and magnitude of depth of sequencing experiment (Tarazona et al., Genome Research, 21:2213-2223, 2011).

Determining the relative changes in frequency of expression of RNA biomarkers specific for prostate cancer allows detection of prostate cancers, distinguishing prostate cancers from benign prostate hypertrophy (BPH) and prostatitis, and detection of prostate cancers in asymptomatic men whose prostate cancer may produce low levels of PSA, with high sensitivity and specificity.

In one aspect, the present disclosure provides methods for detecting the presence of a disorder in a subject, comprising: (a) determining the relative frequency of expression of a plurality of RNA biomarkers in the biological sample, wherein the frequency of expression is determined using next generation sequencing of an amplicon cDNA library prepared using a plurality of oligonucleotide primers specific for the plurality of RNA biomarkers; and (b) comparing the relative frequency of expression of the plurality of RNA biomarkers in the biological sample with predetermined threshold values, wherein increased or decreased relative frequency of expression of at least two or more of the RNA biomarkers in the biological sample indicates the presence of the disorder in the subject.

In certain embodiments, the amplicon cDNA library is prepared by: (a) isolating total RNA from the biological sample; (b) generating first strand cDNA from the total RNA using a plurality of first oligonucleotide primers specific for the plurality of RNA biomarkers; (c) synthesizing second strand cDNA to provide double-stranded cDNA; (d) adding at least one sequencing adapter to the double-stranded cDNA; and (e) amplifying the double-stranded cDNA to provide the amplicon cDNA library. In one embodiment, the double-stranded cDNA is amplified by polymerase chain reaction using a plurality of oligonucleotide primer pairs specific for the plurality of RNA biomarkers after step (c) and prior to step (d).

In an alternative embodiment, the amplicon cDNA library is prepared by: (a) isolating total RNA from the biological sample; (b) preparing first strand cDNA to provide single-stranded cDNA; (c) amplifying the single-stranded cDNA by polymerase chain reaction using a plurality of oligonucleotide primer pairs specific for the plurality of RNA biomarkers to provide amplified double-stranded cDNA; (d) adding at least one sequencing adapter to the amplified double-stranded cDNA; and (e) further amplifying the amplified double-stranded cDNA using primers specific for the at least one sequencing adapter to provide the amplicon cDNA library.

In certain embodiments, the disorder is prostate cancer and the relative frequency of expression of the plurality of RNA biomarkers is determined using: expression levels of the plurality of RNA biomarkers in an adjacent prostate gland sample from the test subject; expression levels of the plurality of RNA biomarkers in a prostate gland sample from a different, healthy, subject; expression levels of the plurality of RNA biomarkers in a sample of prostatectomy gland tissue from a prostatectomy sample that did not show primary tumors upon histological examination; a reference standard established using expression levels of the plurality of RNA biomarkers in a plurality of adjacent prostate gland samples obtained from a plurality of different subjects with the same Gleason scores as the test subject; a reference standard established using expression levels of the plurality of RNA biomarkers in a plurality of adjacent prostate gland samples obtained from a plurality of different subjects with different Gleason scores from the subject; and/or a reference standard established using expression levels of the plurality of RNA biomarkers in a sample of normal human epithelial cells.

In another aspect, the present disclosure provides method for monitoring progression of a disorder in a subject, comprising: (a) determining the relative frequency of expression of a plurality of RNA biomarkers simultaneously in a first biological sample obtained from the subject at a first time point, and determining the relative frequency of expression of the plurality of RNA biomarkers simultaneously in a second biological sample obtained from the subject at a second, subsequent, time point, wherein the relative frequency of expression is determined using next generation sequencing of an amplicon cDNA library prepared using a plurality of oligonucleotide primers specific for the plurality of RNA biomarkers; and (b) comparing the relative frequency of expression of the plurality of RNA biomarkers in the first and second biological samples with a predetermined threshold value, wherein an increase or decrease in the relative frequency of expression of the plurality of RNA biomarkers in the biological sample at the second time point compared to the relative frequency of expression of the plurality of RNA biomarkers at the first time point indicates progression of the disorder in the subject. The amplicon cDNA library is prepared and the relative frequency expression is determined as described above.

In yet another aspect, methods for identifying a subject at risk of developing metastatic cancer or at risk of cancer recurrence, are provided, such methods comprising: (a) determining the relative frequency of expression of a plurality of RNA biomarkers simultaneously in a biological sample obtained from the subject, wherein the frequency of expression is determined using next generation sequencing of an amplicon cDNA library prepared using a plurality of oligonucleotide primers specific for the plurality of RNA biomarkers; and (b) comparing the relative frequency of expression of the plurality of RNA biomarkers in the biological sample with a predetermined threshold value, wherein increased or decreased relative frequency of expression of at least two of the plurality of RNA biomarkers in the biological sample relative to the predetermined threshold value indicates that the subject is at risk of developing metastatic cancer or at risk of cancer recurrence. Again, the amplicon cDNA library is prepared and the relative frequency expression is determined as described above.

In a further aspect, methods for detecting the presence of prostate cancer in a subject are provided that comprise: (a) determining the relative frequency of expression of a plurality of RNA biomarkers simultaneously in a biological sample obtained from the subject, wherein the plurality of RNA biomarkers is selected from the group consisting of: RNA sequences corresponding to DNA sequences provided in SEQ ID NO: 1-75, 235-292, 327-351, 418 and 419, and wherein the frequency of expression is determined using next generation sequencing of an amplicon cDNA library prepared using a plurality of oligonucleotide primers specific for the plurality of RNA biomarkers; (b) comparing the relative frequency of expression of the plurality of RNA biomarkers in the biological sample with a predetermined threshold value; and (c) determining the presence of prostate cancer if there is an increased or decreased relative frequency of expression of at least one RNA biomarker corresponding to a DNA sequence selected from the group consisting of SEQ ID NO: 1-71, 235-287, 327-340, 343-351, 418 and 419 compared to the predetermined threshold value.

In a related aspect, the present disclosure provides methods for monitoring progression of prostate cancer in a subject, comprising: (a) determining the relative frequency of expression of a plurality of RNA biomarkers simultaneously in a biological sample obtained from the subject at a first time point, and determining the relative frequency of expression of the plurality of RNA biomarkers simultaneously in a biological sample obtained from the subject at a second, subsequent, time point, wherein the plurality of RNA biomarkers is selected from the group consisting of: RNA sequences corresponding to DNA sequences provided in SEQ ID NO: 1-75, 235-292, 327-351, 418 and 419, and wherein the relative frequency of expression is determined using next generation sequencing of an amplicon cDNA library prepared using a plurality of oligonucleotide primers specific for the plurality of RNA biomarkers; (b) comparing the relative frequency of expression of the plurality of RNA biomarkers in the biological sample with a predetermined threshold value; and (c) determining the progression of prostate cancer in the subject if the relative frequency of expression of at least one RNA biomarker corresponding to a DNA sequence selected from the group consisting of SEQ ID NO: 1-71, 235-287, 327-340, 343-351, 418 and 419 is increased or decreased at the second time point compared to the relative frequency of expression of the RNA biomarker at the first time point.

In yet a further aspect, the present disclosure provides methods for predicting a likelihood of the presence of prostate cancer in a test subject that comprise: (a) measuring the expression levels of a plurality of RNA biomarkers in a biological sample obtained from the subject, wherein the plurality of RNA biomarkers comprises at least three RNA biomarkers selected from the group consisting of: RNA sequences corresponding to DNA sequences provided in SEQ ID NO: 1-75, 235-292, 327-351, 418 and 419; (b) comparing the expression level of each of the plurality of RNA biomarkers in the biological sample with a predetermined reference standard for the RNA biomarker; and (c) predicting the likelihood of the presence of prostate cancer in the subject based on a comparison of the expression level of each of the plurality of RNA biomarkers with the predetermined reference standard for the RNA biomarker. In certain embodiments, expression levels of the plurality of RNA biomarkers that are above or below the predetermined reference standards are indicative of the presence of prostate cancer in the subject. In specific embodiments, the plurality of RNA biomarkers comprises, or consists of, at least three (for example, three, four, five, six, seven or more) RNA biomarkers corresponding to DNA sequences selected from the group consisting of: (i) SEQ ID NO: 41 (PSMA), SEQ ID NO: 49 (TDRD1), SEQ ID NO: 241 (Clorf64), SEQ ID NO: 248 (CST4), and SEQ ID NO: 261 (PCA3); (ii) SEQ ID NO: 1 (ADM), SEQ ID NO: 7 (C15orf48), SEQ ID NO: 25 (KLK3), SEQ ID NO: 39 (PLA2G7), SEQ ID NO: 44 (SLC10A7), SEQ ID NO: 51 (TMC5), SEQ ID NO: 57 (AZGP1), SEQ ID NO: 235 (ACSM3), and SEQ ID NO: 248 (CST4); (iii) SEQ ID NO: 1 (ADM), SEQ ID NO: 7 (C15orf48), SEQ ID NO: 11 (ETV4), SEQID NO: 49 (TDRD1), SEQ ID NO: 51 (TMC5), SEQ ID NO: 57 (AZGP1), and SEQID NO: 254 (GPR116); or (iv) SEQ ID NO: 8 (CRISP3), SEQ ID NO: 12 (F5), SEQ ID NO: 22 (HPN), SEQ ID NO: 35 (PEX10), SEQ ID NO: 39 (PLA2G7), SEQ ID NO: 44 (SLC10A7), SEQ ID NO: 59 (CSRP1), SEQ ID NO: 248 (CST4), SEQ ID NO: 254 (GPR116), SEQ ID NO: 261 (PCA3), and SEQ ID NO: 286 (SLC22A17).

In a related aspect, methods for generating a prostate cancer differential expression profile for a subject are provided that comprise: (a) measuring expression levels of a plurality of RNA biomarkers in a biological sample obtained from the subject, wherein the plurality of RNA biomarkers comprises at least three RNA biomarkers selected from the group consisting of: RNA sequences corresponding to DNA sequences provided in SEQ ID NO: 1-75, 235-292, 327-351, 418 and 419; (b) determining whether expression of each of the plurality of RNA biomarkers in the biological sample is up-regulated or down-regulated relative to a predetermined reference standard for each of the plurality of RNA biomarkers; and (c) generating a prostate cancer differential expression profile for the test subject. In certain embodiments, the prostate cancer differential expression profile is generated, or provided, in a format selected from the group consisting of: a database, an electronic display, a paper report, a text document, a graphic display and a digital format.

Biological samples that can be effectively employed in the disclosed methods include, but are not limited to, urine, blood, serum, cell lines, peripheral blood mononuclear cells (PBMCs), biopsy tissue and prostatectomy tissue.

In certain embodiments, the disclosed methods comprise determining the expression level of a plurality of RNA biomarkers corresponding to a plurality of polynucleotide biomarkers selected from the group consisting of those listed in Tables 1, 2, 3 and 4. Panels and kits comprising a plurality (for example, two, three, four, five, six, seven, eight, nine, ten or more) of such isolated RNA biomarkers are also provided.

Oligonucleotide primers that can be employed in the methods disclosed herein include, but are not limited to, those provided in SEQ ID NO: 76-232, 293-326 and 352-417. In certain embodiments, the methods disclosed herein include detecting the relative frequency of expression of a RNA biomarker comprising an RNA sequence that corresponds to a DNA sequence of SEQ ID NO: 1-75, 235-287, 327-351, 418 and 419 or a variant thereof, as defined herein. Those of skill in the art will appreciate that the RNA sequences for the disclosed RNA biomarkers are identical to the cDNA sequences disclosed herein except for the substitution of thymine (T) residues with uracil (U) residues.

In a further aspect, the present disclosure provides an oligonucleotide primer comprising, or consisting of, a sequence selected from the group consisting of SEQ ID NO: 76-232, 293-326 and 352-417, and variants thereof. In certain embodiments, such oligonucleotide primers have a length equal to or less than 30 nucleotides. In addition to being effective in the diagnostic methods disclosed herein, the disclosed oligonucleotide primers can be effectively employed in other methods for diagnosing the presence of, and/or monitoring the progression of, prostate cancer that are well known to those of skill in the art, including quantitative real time PCR and small scale oligonucleotide microarrays.

In a related aspect, the present disclosure provides panels and kits containing a plurality (for example at least two, three, four, five or more) of the oligonucleotide primers disclosed herein. Such panels and kits can be effectively employed in the diagnosis, prognosis and monitoring of prostate cancers. In certain embodiments, the disclosed panels and kits further include at least one oligonucleotide primer that is specific for a reference gene. Examples of reference genes and their corresponding primers are provided in Table 3 below. The oligonucleotide primers included in the disclosed kits can be packaged individually in vials, in combination in containers and/or in multi-container units. Such kits can be advantageously used for carrying out the methods disclosed herein and optionally include instructions for the use of the oligonucleotide primers, for example in the disclosed methods, and/or a device for obtaining or providing a biological sample.

In yet a further aspect, methods for establishing a reference standard for a biomarker for use in diagnosis, prognosis and/or monitoring of prostate cancer in a subject are provided, the methods comprising determining the expression level of the biomarker in at least one biological sample selected from the group consisting of: (a) an adjacent prostate gland sample obtained from the test subject; (b) a plurality of prostate gland samples from different, healthy subjects; (c) a plurality of samples of prostatectomy gland tissue from prostatectomy samples that did not show primary tumors upon histological examination; (d) a plurality of adjacent prostate gland samples obtained from a plurality of different subjects with the same Gleason scores as the test subject; (e) a plurality of adjacent prostate gland samples obtained from a plurality of different subjects with different Gleason scores from the test subject; and (f) a sample of normal human epithelial cells.

In one embodiment, methods for establishing a reference standard for a RNA biomarker for use in diagnosing the presence of prostate cancer in a test subject are provided that comprise: (a) measuring the expression level of the RNA biomarker in at least two (for example, two, three, four, five, six or more) biological samples selected from the group consisting of: (i) prostate gland samples obtained from different, healthy, subjects; (ii) prostatectomy gland tissue from prostatectomy samples that do not show primary tumors upon histological examination; (iii) adjacent prostate gland samples obtained from different subjects with the same Gleason scores as the test subject; and (iv) adjacent prostate gland samples obtained from different subjects with different Gleason scores from the test subject; (b) determining the mean and the standard deviation of the expression level in the at least one biological sample; and (c) determining a lower end of a normal range of expression of the biomarker as the mean minus two standard deviations, and determining an upper end of a normal range of expression of the biomarker as the mean plus two standard deviations, thereby establishing the reference standard. Methods for determining whether a biomarker is differentially expressed in a biological sample obtained from a test subject that employ reference standards established using such methods are also provided.

### BRIEF DESCRIPTION OF THE DRAWINGS

FIG. 1 shows four adaptations to conventional NGS technology that are employed in the disclosed methods.

FIG. 2 shows the independent filtering function plot used by DESeq2 allowing identification of lowest expressed genes across all samples that show no significant p-value.

FIG. 3 shows the differential expression profile of RNA biomarkers from the comparison of subject's tumor with a Gleason score of 5 (3+2) versus the subject's own adjacent gland: Up-regulation in black, down-regulation in grey and no differential expression in white.

FIG. 4 shows the establishment of a reference standard.

FIG. 5 shows the comparison of primary tumor (PT) samples to a reference standard (R).

FIGS. 6A and 6B show the differential expression profile of RNA biomarkers from the comparison of a subject's adjacent gland (FIG. 6A) and tumor with a Gleason score of 5 (4+3) (FIG. 6B) versus the reference standard (Rv1): Up-regulation in black, down-regulation in grey and no differential expression in white.

### DEFINITIONS

As used herein, the term “biomarker” refers to a molecule that is associated either quantitatively or qualitatively with a biological change. Examples of biomarkers include polypeptides, proteins, fragments of a polypeptide or protein; polynucleotides, such as a gene product, RNA or RNA fragment; and other body metabolites.

As used herein, the term “RNA biomarker” or “gene transcript biomarker” refers to a RNA molecule produced by transcription of a gene that is associated either quantitatively or qualitatively with a biological change.

As used herein the term “RNA sequence corresponding to a DNA sequence” refers to a sequence that is identical to the DNA sequence except for the substitution of all thymine (T) residues with uracil (U) residues.

As used herein, the term “oligonucleotide specific for a biomarker” refers to an oligonucleotide that specifically hybridizes to a polynucleotide biomarker (such as an RNA biomarker) or a polynucleotide encoding a polypeptide biomarker, and that does not significantly hybridize to unrelated polynucleotides. In certain embodiments, the oligonucleotide hybridizes to a gene, a gene fragment or a gene transcript. In specific embodiments, the oligonucleotide hybridizes to the polynucleotide of interest under stringent conditions, such as, but not limited to, prewashing in a solution of 6×SSC, 0.2% SDS; hybridizing at 65° C., 6×SSC, 0.2% SDS overnight; followed by two washes of 30 minutes each in 1×SSC, 0.1% SDS at 65° C. and two washes of 30 minutes each in 0.2×SSC, 0.1% SDS at 65° C.

As used herein the term “oligonucleotide primer pair” refers to a pair of oligonucleotide primers that span an intron in the cognate gene transcript biomarker.

As used, herein the term “polynucleotide(s),” refers to a single or double-stranded polymer of deoxyribonucleotide or ribonucleotide bases and includes deoxyribonucleic acid (DNA) and corresponding ribonucleic acid (RNA) molecules, including hnRNA, mRNA and non-coding RNA molecules, both sense and anti-sense strands, and includes cDNA, genomic DNA and recombinant DNA, as well as wholly or partially synthesized polynucleotides. An hnRNA molecule contains introns and corresponds to a DNA molecule in a generally one-to-one manner. An mRNA molecule corresponds to an hnRNA and DNA molecule from which the introns have been excised. A non-coding RNA is a functional RNA molecule that is not translated into a protein, although in some circumstances non-coding RNA can be coding and vice versa.

As used herein, the term “amplicon” refers to pieces of DNA that have been synthesized using amplification techniques such as, but not limited to, polymerase chain reaction (PCR).

As used herein, the term “subject” refers to a mammal, preferably a human, who may or may not have a disorder of interest, such as prostate cancer. Typically, the terms “subject” and “patient” are used interchangeably herein in reference to a human subject.

As used herein, the term “healthy subject” refers to a subject who is not inflicted with a disorder of interest.

As used herein in connection with prostate cancer, the term “healthy male” refers to a male who has an undetectable PSA level in serum or non-rising PSA levels up to 1 ng/ml, no evidence of prostate gland abnormality following a DRE and no clinical symptoms of a prostatic disorder.

As used herein in connection with prostate cancer, the term “asymptomatic male” refers to a male who has a PSA level in serum of greater than 4 ng/ml, which is considered indicative of prostate cancer, but whose DRE is inconclusive and who has no clinical symptoms of disease.

The term “benign prostate hypertrophy” (BPH) refers to a prostatic disease with a non-malignant growth of epithelial cells in the prostate gland and the term “prostatitis” refers to another prostatic disease of the prostate, usually due to a microbial infection of the prostate gland. Both BPH and prostatitis can result in increased PSA levels.

As used herein, the term “metastatic prostate cancer” refers to prostate cancer which has spread beyond the prostate gland to a distant site, such as lymph nodes or bone.

As used herein, the term “indolent cancer” or “insignificant cancer” refers to a cancer that is unlikely to progress to clinical significance in the absence of treatment. Such cancers are generally low-grade, small-volume and organ-confined.

As used herein the term “aggressive cancer” or “significant cancer” refers to a cancer that is likely to progress to clinical significance, including metastatic disease and ultimately death, in the absence of treatment.

As used herein, the term “watchful waiting” refers to monitoring of a patient's condition without giving any treatment until symptoms appear or change. Watchful waiting is typically employed with patients who have an indolent cancer.

As used herein, the term “biopsy tissue” refers to a sample of tissue (e.g., prostate tissue) that is removed from a subject for the purpose of determining if the sample contains cancerous tissue. The biopsy tissue is then examined (e.g., by microscopy) for the presence or absence of cancer.

As used herein, the term “prostatectomy” refers to the surgical removal of the prostate gland.

The term “sample” as used herein refers to a sample, specimen or culture obtained from any source. Biological samples include blood products (such as plasma, serum and whole blood), urine, saliva and the like. Biological samples also include tissue samples, such as biopsy tissues or pathological tissues that have previously been fixed (e.g., formalin, snap frozen, cytological processing, etc.).

As used herein, the term “predetermined threshold value” of expression of a gene transcript biomarker, or RNA biomarker, refers to the level of expression of the same biomarker in: (a) one or more corresponding control/normal samples obtained from the same subject; (b) one or more control/normal samples obtained from normal, or healthy, subjects, e.g. from males who do not have prostate cancer; or (c) a corresponding reference standard.

As used herein, the term “altered frequency of expression” of a gene transcript in a test biological sample refers to a frequency that is either below or above the predetermined threshold value of expression for the same gene transcript in a control sample and thus encompasses either high (increased) or low (decreased) expression levels.

As used herein, the term “relative frequency of expression” or “differential expression profile” refers to the frequency of expression of a gene transcript biomarker or RNA biomarker in a test biological sample relative to the frequency of expression of the same biomarker in a corresponding reference standard, a control/normal sample or a group of control/normal samples obtained either from the same subject or from normal, or healthy, subjects, (e.g., from males who do not have prostate cancer).

As used herein, the term “prognosis” or “providing a prognosis” for a disorder, such as prostate cancer, refers to providing information regarding the likely impact of the presence of prostate cancer (e.g., as determined by the diagnostic methods disclosed herein) on a subject's future health (e.g., the risk of metastasis).

As used herein, the term “adjacent prostate gland sample” refers to a prostate gland sample that is located adjacent to a prostate cancer lesion and that is believed to be non-cancerous based on histological examination.

The Gleason Grading system is a system of grading prostate tumor based on its microscopic appearance that is used to help evaluate the prognosis of men with prostate cancer. Gleason scores comprise grades of the two most common tumor patterns in a prostate tumor sample.

### DETAILED DESCRIPTION

As outlined above, the present disclosure provides methods for detecting the presence or absence of a disorder, for example a cancer such as prostate cancer, in a subject, determining the stage of the disorder and/or the phenotype of the disorder, monitoring progression of the disorder, and/or monitoring treatment of the disorder by determining the frequency of expression of specific gene transcript biomarkers, or RNA biomarkers, in a biological sample obtained from the subject. The methods disclosed herein employ one or more modifications of standard NGS protocols. The disclosed methods employ oligonucleotides specific for multiple gene transcript biomarkers in combination with NGS technology to perform parallel amplicon synthesis and sequencing, and thereby determine the relative frequency of expression of the gene transcript biomarkers in a sample. Such methods have significant advantages over other technologies typically employed to determine expression levels of polynucleotide biomarkers, including improved accuracy, reproducibility and throughput, and can be employed to accurately and simultaneously determine the frequency of expression of a multitude of gene transcript biomarkers across a large number of samples.

In specific embodiments, such methods use oligonucleotides specific for one or more biomarkers selected from those shown in Tables 1, 2 and 4. In certain embodiments, such methods further employ one or more reference genes selected from those shown in Table 3.

In one embodiment, the disclosed methods comprise determining the relative frequency of expression levels of at least two, three, four, five, six, seven, eight, nine, ten or more gene transcript biomarkers, or RNA biomarkers, selected from the group consisting of: SEQ ID NO: 1-75, 235-292, 327-351, 418 and 419 in a biological sample taken from a subject, and comparing the relative frequency of expression of the biomarkers with predetermined threshold values.

The disclosed methods can be employed to diagnose the presence of prostate cancer in asymptomatic subjects; subjects with early stage prostate cancer; subjects who have had surgery to remove the prostate (radical prostatectomy); subjects who have had radiation treatment for prostate cancer; subjects who are undergoing, or have completed, androgen ablation therapy; subjects who have become resistant to hormone ablation therapy; and/or subjects who are undergoing, or have had, chemotherapy.

In certain embodiments, the gene transcript biomarkers disclosed herein appear in subjects with prostate cancer at levels that are at least two and a half log₂ fold higher or lower than, or at least two standard deviations above or below, the mean level in a reference standard.

In one embodiment, the up- or down-regulation of one RNA biomarker may be associated with the up- or down-regulation of a specific set of two or more RNA biomarkers indicative of a specific activation state of the androgen receptor.

All of the biomarkers and oligonucleotides disclosed herein are isolated and purified, as those terms are commonly used in the art. Preferably, the biomarkers and oligonucleotides are at least about 80% pure, more preferably at least about 90% pure, and most preferably at least about 99% pure.

In certain embodiments, the oligonucleotides employed in the disclosed methods specifically hybridize to a variant of a polynucleotide biomarker disclosed herein. As used herein, the term “variant” comprehends nucleotide or amino acid sequences different from the specifically identified sequences, wherein one or more nucleotides or amino acid residues is deleted, substituted, or added. Variants may be naturally occurring allelic variants, or non-naturally occurring variants. Variant sequences (polynucleotide or polypeptide) preferably exhibit at least 80%, 85%, 90%, 95%, 96%, 97%, 98% or 99% identity to a sequence disclosed herein. The percentage identity is determined by aligning the two sequences to be compared as described below, determining the number of identical residues in the aligned portion, dividing that number by the total number of residues in the inventive (queried) sequence, and multiplying the result by 100.

In addition to exhibiting the recited level of sequence identity, variants of the disclosed biomarkers are preferably themselves expressed in subjects with prostate cancer at a frequency that are higher or lower than the levels of expression in normal, healthy individuals.

Polypeptide and polynucleotide sequences may be aligned, and percentages of identical amino acids or nucleotides in a specified region may be determined against another polypeptide or polynucleotide sequence, using computer algorithms that are publicly available. The percentage identity of a polynucleotide or polypeptide sequence is determined by aligning polynucleotide and polypeptide sequences using appropriate algorithms, such as BLASTN or BLASTP, respectively, set to default parameters; identifying the number of identical nucleic or amino acids over the aligned portions; dividing the number of identical nucleic or amino acids by the total number of nucleic or amino acids of the polynucleotide or polypeptide of the present invention; and then multiplying by 100 to determine the percentage identity.

Two exemplary algorithms for aligning and identifying the identity of polynucleotide sequences are the BLASTN and FASTA algorithms. The alignment and identity of polypeptide sequences may be examined using the BLASTP algorithm. BLASTX and FASTX algorithms compare nucleotide query sequences translated in all reading frames against polypeptide sequences. The FASTA and FASTX algorithms are described in Pearson and Lipman, Proc. Natl. Acad. Sci. USA 85:2444-2448, 1988; and in Pearson, Methods in Enzymol. 183:63-98, 1990. The FASTA software package is available from the University of Virginia, Charlottesville, Va. 22906-9025. The FASTA algorithm, set to the default parameters described in the documentation and distributed with the algorithm, may be used in the determination of polynucleotide variants. The readme files for FASTA and FASTX Version 2.0x that are distributed with the algorithms describe the use of the algorithms and describe the default parameters.

The BLASTN software is available on the NCBI anonymous FTP server and is available from the National Center for Biotechnology Information (NCBI), National Library of Medicine, Building 38A, Room 8N805, Bethesda, Md. 20894. The BLASTN algorithm Version 2.0.6 [Sep. 10, 1998] and Version 2.0.11 [Jan. 20, 2000] set to the default parameters described in the documentation and distributed with the algorithm, is preferred for use in the determination of variants according to the present invention. The use of the BLAST family of algorithms, including BLASTN, is described at NCBI's website and in the publication of Altschul, et al., “Gapped BLAST and PSI-BLAST: a new generation of protein database search programs,” Nucleic Acids Res. 25:3389-3402, 1997.

Variant sequences generally differ from the specifically identified sequence only by conservative substitutions, deletions or modifications. As used herein with regards to amino acid sequences, a “conservative substitution” is one in which an amino acid is substituted for another amino acid that has similar properties, such that one skilled in the art of peptide chemistry would expect the secondary structure and hydropathic nature of the polypeptide to be substantially unchanged. In general, the following groups of amino acids represent conservative changes: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his. Variants may also, or alternatively, contain other modifications, including the deletion or addition of amino acids that have minimal influence on the antigenic properties, secondary structure and hydropathic nature of the polypeptide. For example, a polypeptide may be conjugated to a signal (or leader) sequence at the N-terminal end of the protein which co-translationally or post-translationally directs transfer of the protein. The polypeptide may also be conjugated to a linker or other sequence for ease of synthesis, purification or identification of the polypeptide (e.g., poly-His), or to enhance binding of the polypeptide to a solid support. For example, a polypeptide may be conjugated to an immunoglobulin Fc region.

In another embodiment, variant polypeptides are encoded by polynucleotide sequences that hybridize to a disclosed polynucleotide under stringent conditions. Stringent hybridization conditions for determining complementarity include salt conditions of less than about 1 M, more usually less than about 500 mM, and preferably less than about 200 mM. Hybridization temperatures can be as low as 5° C., but are generally greater than about 22° C., more preferably greater than about 30° C., and most preferably greater than about 37° C. Longer DNA fragments may require higher hybridization temperatures for specific hybridization. Since the stringency of hybridization may be affected by other factors such as probe composition, presence of organic solvents and extent of base mismatching, the combination of parameters is more important than the absolute measure of any one alone. An example of “stringent conditions” is prewashing in a solution of 6×SSC, 0.2% SDS; hybridizing at 65° C., 6×SSC, 0.2% SDS overnight; followed by two washes of 30 minutes each in 1×SSC, 0.1% SDS at 65° C. and two washes of 30 minutes each in 0.2×SSC, 0.1% SDS at 65° C.

The expression levels of one or more gene transcript biomarkers, or RNA biomarkers, in a biological sample can be determined, for example, using one or more oligonucleotides that are specific for the gene transcript or RNA biomarker. RNA is isolated from the biological sample and the frequency of expression of a gene transcript or RNA biomarker of interest is determined as described below using oligonucleotides specific for the gene transcript or RNA biomarker of interest in combination with modified NGS technology.

In other embodiments, the levels of mRNA corresponding to a biomarker disclosed herein can be detected using oligonucleotides in Southern hybridizations, in situ hybridizations, or quantitative real-time PCR amplification (RT-qPCR). Solid phase substrates, or carriers, that can be effectively employed in such assays are well known to those of skill in the art and include, but are not limited to, microporous membranes constructed, for example, of nitrocellulose, nylon, polyvinylidene difluoride, polyester, cellulose acetate, mixed cellulose esters and polycarbonate. Suitable microporous membranes include, for example, those described in US Patent Application Publication no. US2010/0093557A1. Methods for performing such assays are well known to those of skill in the art.

The present disclosure further provides methods employing a plurality of oligonucleotides that are specific for a plurality of the prostate cancer gene transcript biomarkers disclosed herein. The oligonucleotides employed in the disclosed methods are generally single-stranded molecules, such as synthetic antisense molecules or cDNA fragments, and are, for example, 6-60 nt, 15-30 nt or 20-25 nt in length.

Oligonucleotides specific for a polynucleotide, or gene transcript, biomarker disclosed herein are prepared using techniques well known to those of skill in the art. For example, oligonucleotides can be designed using known computer algorithms to identify oligonucleotides of a defined length that are unique to the polynucleotide, have a GC content within a range suitable for hybridization, and lack predicted secondary structure that may interfere with hybridization. Oligonucleotides can be synthesized using methods well known to those in the art. In specific embodiments, the oligonucleotides employed in the disclosed methods and compositions are selected from the group consisting of: SEQ ID NO: 76-232, 293-326 and 352-417.

For tests involving alterations in RNA expression levels, it is important to ensure adequate standardization. Accordingly, in tests such as the adapted NGS technology disclosed herein, RT-qPCR or small scale oligonucleotide microarrays, at least one reference gene is employed. Reference genes that can be employed in such methods include, but are not limited to, those listed in Table 3 below.

In one example below, the establishment of a reference standard is described. This approach was developed to approximate the level and normal biological variation of expression of the biomarkers in non-cancerous prostate tissue. The reference standard is built using the most ‘normal’ glandular samples available, including samples from subjects with no confirmed tumor or with low Gleason score tumors (5 and 6).

In other examples, the differential expression of RNA biomarkers in samples derived from cancerous tissue with a Gleason score of 5 and 6 (referred to herein as Groups I and II) is described by comparison to the reference standard described above, together with the differential expression of RNA biomarkers in samples derived from cancerous tissue with a Gleason score of 3+4 (Group III), 4+3 (Group IV) and 8-10 (Group V). The segregation between Groups I and II on the one hand and Groups III, IV and V on the other reflects the segregation of the tumors into those in which it is unclear whether they will progress or remain indolent (Groups I and II), and those that are highly likely to progress and become life threatening (Groups III, IV and V).

Also described is a method for relating gene transcript changes to a number of genes closely linked to the androgen receptor. This analytical approach creates a personalized integrative gene network linking the RNA biomarker expression profile of each analyzed subject to the androgen receptor and other key regulators of prostate cancer initiation and development. This integrative analytical method is of clinical relevance as it allows a rapid characterization of the large amount of data generated by NGS sequencing of amplicon libraries from each tissue sample and can serve as an interpretation tool to associate the expression profiles of multiple RNA biomarkers to specific diagnosis and prognosis of prostate cancer.

The following examples are intended to illustrate, but not limit, this disclosure.

### EXAMPLES

### Materials and Methods

### RNA Extraction

Archived formalin fixed paraffin embedded (FFPE) prostatectomy tissue was collected from Diagnostic Medical Laboratory NZ Ltd (DML) for Clinical study 1, with permission from donors under the human ethics approval granted by the Southern Health and Disability Ethics Committee (reference 12/STH/62 dated 22 Feb. 2013).

FFPE blocks were reviewed by a clinical histopathologist, and a tumor and histologically adjacent region deemed “normal” were identified for each subject. These identified areas were then excised and reset in paraffin. Approximately fifteen freshly cut sections at a thickness of ten microns were then processed using a Qiagen RNeasy FFPE kit (Cat No: 74404, 73504) to extract RNA. In all extractions the method used for the deparaffinized step was the original method from Cat No: 74404 kit, and the remainder of the protocol was performed following the manufacturer's instructions.

RNA purity was assessed on the NanoDrop 2000 spectrophotometer (Thermo Scientific), and the RNA concentration was determined using the Qubit® 2.0 Fluorometer RNA assay kit (Life Technologies). RNA integrity was evaluated using the RNA 6000 NanoAssay for the Agilent Bioanalyser 2100 (Agilent Technologies, Santa Clara, Calif.).

### RNA Biomarker Amplicon Production

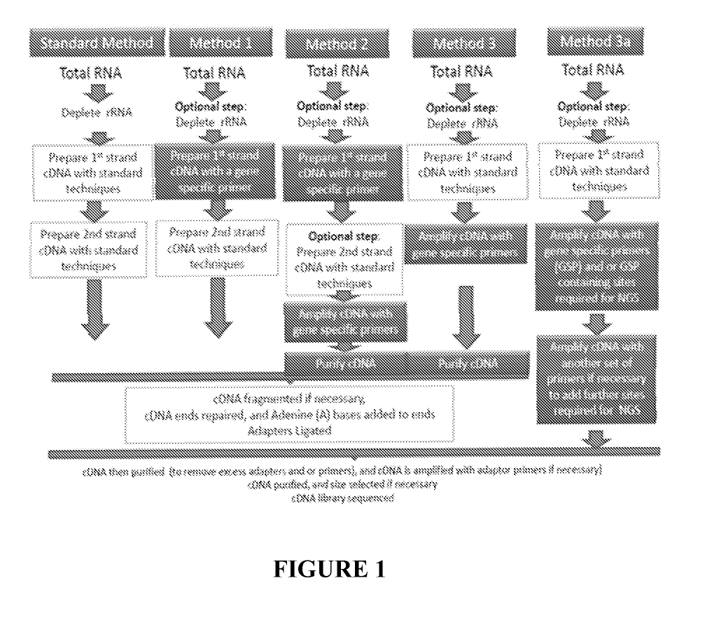

The relative frequency of expression of specific RNA biomarkers was determined using the isolated RNA in one or more of the four methods described below and summarized in FIG. 1. Each of these methods includes at least one modification of conventional NGS technologies. Conventional NGS technologies are well known to those of skill in the art and are described, for example, in Wang et al. (Nat. Rev. Genet. (2009) 10:57-63), and Marguerat and Bahler (Cell. Mol. Life Sci. (2010) 67:569-579).

### Method 1

In a first method, sequence specific priming is employed during the generation of first strand cDNA. An optional first step in this method is to deplete the total RNA of rRNA using an industry-provided kit, if necessary. An industry-provided first strand cDNA kit is used to combine total RNA (or rRNA-depleted total RNA) with at least one strand specific oligonucleotide primer (i.e. an oligonucleotide primer specific for the RNA biomarker of interest) and generate first strand cDNA according to the manufacturer's protocol. Second strand cDNA is then synthesized in an unbiased manner using standard techniques. The resulting double-stranded cDNA is fragmented if necessary using standard methods, and the cDNA ends are repaired using standard methods in which any overhangs at the cDNA ends are converted into blunt ends using T4 DNA polymerase. An overhanging adenine (A) base is added to the 3′ end of the blunt DNA fragments by the use of Klenow fragment to assist with ligation of adapters required for the sequencing process. The adapters are ligated to the ends of the cDNA fragments using standard procedures, and then the cDNA fragments are run on a gel for purification and removal of excess adapters. The cDNA is amplified using adapter primers, purified, denatured and further diluted for cluster generation and sequencing, for example on a HiSeq2000 according to Illumina Corporation's standard protocols (208 cycles sequencing program, paired-end with indexing). The cDNA library is sequenced, and the relative frequency of expression of the specific RNA biomarkers in cancer patients and healthy controls is determined.

### Method 2

As in method 1, sequence specific priming is employed during the generation of first strand cDNA. This is achieved using an industry provided first strand cDNA kit and at least one strand specific oligonucleotide primer to generate first strand cDNA from total RNA (or rRNA depleted total RNA if necessary) according to the manufacturer's protocol. The second strand cDNA can either be prepared in an unbiased manner using standard techniques, or it can be directly amplified using a set of specific oligonucleotide primers (i.e. oligonucleotide primers specific for the RNA biomarkers of interest) to amplify a specific set of PCR amplicons by either primer limited or cycle limited PCR. In preferred embodiments, the oligonucleotide primer employed to generate the first strand cDNA can be the same as one of the pair of oligonucleotide primers used to amplify the double-stranded cDNA. The cDNA is then purified via a cleanup procedure to remove excess PCR reagents. The cDNA is fragmented if necessary using standard methods, and the cDNA ends are repaired using standard methods in which any overhangs at the cDNA ends are converted into blunt ends using T4 DNA polymerase. An overhanging adenine (A) base is added to the 3′ end of the blunt DNA fragments by the use of Klenow fragment to assist with ligation of adapters required for the sequencing process. The adapters are ligated to the ends of the cDNA fragments using standard procedures, and the cDNA fragments are then purified to remove excess adapters. The cDNA is amplified using adapter primers, purified, denatured and further diluted for cluster generation and sequencing, for example on a HiSeq2000 according to Illumina Corporation's standard protocols (208 cycles sequencing program, paired-end with indexing). The cDNA library is sequenced and the relative frequency of expression of the specific RNA biomarkers in cancer patients and healthy controls is determined.

### Method 3

This method employs total RNA or rRNA-depleted RNA if necessary. The first strand cDNA is synthesized using standard methods. The first strand cDNA is then directly amplified using a set of specific oligonucleotide primers (i.e. oligonucleotide primers specific for the RNA biomarkers of interest) to amplify a specific set of PCR amplicons using either primer limited or cycle limited PCR. The cDNA is purified via a cleanup procedure to remove excess PCR reagents. The cDNA is fragmented if necessary using standard methods, and the cDNA ends are repaired using standard methods, in which any overhangs at the cDNA ends are converted into blunt ends using T4 DNA polymerase. An overhanging adenine (A) base is added to the 3′ end of the blunt DNA fragments by the use of Klenow fragment to assist with ligation of adapters required for the sequencing process. Adapters are ligated to the ends of the cDNA fragments using standard procedures, and the cDNA is purified to remove excess adapters. The cDNA is then amplified using adapter primers and purified. The cDNA can be size selected via gel electrophoresis using standard methods if necessary. The cDNA library is sequenced, and the relative frequency of expression of the specific RNA biomarkers in cancer patients and healthy controls is determined.

### Method 3a

Method 3a differs from Method 3 in that all sequences necessary for next generation sequencing are incorporated via either a one or two step PCR amplification.

An optional first step in this method is to deplete the total RNA of rRNA using an industry-provided kit, if necessary. The first strand cDNA is then synthesized using standard methods. The first strand cDNA is directly amplified using a set of specific oligonucleotide primers (i.e. oligonucleotide primers specific for the RNA biomarkers of interest) also containing Next Generation Sequencing (NGS) primer sites, using either primer limited or cycle limited PCR. The cDNA is then purified via a cleanup procedure to remove excess PCR reagents, and re-amplified with another set of primers, if necessary, in order to add further sites required for NGS using either primer limited or cycle limited PCR. The cDNA is then purified to remove excess PCR reagents and, if necessary, is again amplified using adapter primers and purified. The cDNA is then denatured and further diluted for cluster generation and sequencing, for example on a HiSeq2000 according to Illumina Corporation's standard protocols (208 cycles sequencing program, paired-end with indexing). The cDNA library is sequenced, and the relative frequency of expression of the specific RNA biomarkers in cancer patients and healthy controls is determined.

### Identification of Prostate Cancer RNA Biomarkers

RNA biomarkers were selected using annotation and analysis of publicly available RNA expression profile data in the NCBI databases GSE6919 and GSE38241 as these data-sets include data from cancer free donors. The NCBI database GSE6919, which was developed at the University of Pittsburgh, contains data from three Affymetrix chips (U95A, U95B and U95C), representing more than 36,000 gene reporters. The database, which has been analyzed by Chandran et al. (BMC Cancer 2005, 5:45; BMC Cancer 2007, 9:64), and Yu et al. (J Clin Oncol 2004, 22:2790-2799) contains RNA profiles from more than 200 individual prostate tumor samples, combined with adjacent “normal” or “healthy” tissues, or prostate tissues from individuals believed to be free of prostate cancer.

The biomarkers shown in Table 1 below form a unique set identified as being over-expressed in subjects with prostate cancer. Similarly, the biomarkers shown in Table 2 form a second unique combination of RNA biomarkers identified as being under-expressed in subjects with prostate cancer.

TABLE 1
RNA Biomarkers with Elevated Expression Levels in Prostate Cancer Patients

| | | | | SEQ | PRIMER | |
|---|---|---|---|---|---|---|
| | GENBANK | | GENE | ID | SEQ ID | PRIMER |
| REPORTER | ACCESSION | GENE DESCRIPTION | SYMBOL | NO: | NOs: | IDs |
| 34777_at | D14874 | Adrenomedullin | ADM | 1 | 76, 77 | ND654, |
| | | | | | | ND655 |
| 38827_at | AF038451 | Anterior gradient 2 | AGR2 | 2 | 78, 79 | ND543, |
| | | homolog | | | | ND544 |
| 37399_at | D17793 | Aldo-keto reductase | AKR1C3 | 3 | 80, 81 | ND498, |
| | | family 1, member C3 | | | | ND499 |
| 41764_at | AA976838 | Apolipoprotein C-I | ApoC1 | 4 | 82, 83 | ND414, |
| | | | | | | ND599 |
| 608_at | M12529 | Apolipoprotein E | ApoE | 5 | 84, 85 | CH350, |
| | | | | | | CH351 |
| 1577_at | M23263 | Androgen receptor | AR | 6 | 86, 87, | ND460, |
| | | | | | 88, 89 | ND461, |
| | | | | | | ND532, |
| | | | | | | ND533 |
| 56999_at | AI625959 | Chromosome 15 open | C15ORF48 | 7 | 90, 91 | CH075, |
| | | reading frame 48 | | | | CH076 |
| 36464_at | X94323 | cysteine-rich secretory | CRISP3 | 8 | 92, 93 | ND536, |
| | | protein 3 | | | | ND537 |
| 40201_at | M76180 | Dopa decarboxylase | DDC | 9 | 94, 95 | CH127, |
| | | | | | | CH128 |
| 37156_at | AF070641 | ets variant gene 1 | ETV1 | 10 | 96, 97 | ND440, |
| | | | | | | ND441 |
| 2084_s_at | D12765 | ets variant gene 4 (E1A | ETV4 | 11 | 98, 99 | ND410, |
| | | enhancer binding | | | | ND411 |
| | | protein, E1AF) | | | | |
| 35245_at | M16967 | F5, Coagulation factor V | F5 | 12 | 100, 101 | ND714, |
| | | | | | | ND715 |
| 36622_at | AI989422 | Fibrinogen | FGG | 13 | 102, 103 | ND442, |
| | | | | | | ND443 |
| 36201_at | D13315 | Glycoxalase 1 | GLO1 | 14 | 104, 105 | CH186, |
| | | | | | | CH187 |
| 39135_at | AB018310 | GRAM domain | GRAMD4 | 15 | 106, 107 | ND484, |
| | | containing 4 | | | | ND589 |
| 48885_at | R61847 | Glutamate receptor, | GRIN3A | 16 | 108, 109 | CH328, |
| | | ionotropic N-methyl-D- | | | | CH329 |
| | | aspartate 3A | | | | |
| 1039_s_at | U22431 | Hypoxia inducible factor 1, | HIF-1A | 17 | 110, 111 | ND700, |
| | | alpha subunit | | | | ND701 |
| 37851_at | AF055019 | Homeodomain | HIPK2 | 18 | 112, 113 | ND612, |
| | | interacting protein | | | | ND613 |
| | | kinase: TF kinase | | | | |
| 32480_at | X07495 | Homeobox C4 | HOXC4 | 19 | 114, 115 | ND422, |
| | | | | | | ND423 |
| 56429_at | AI525822 | Homo sapiens | HN1 | 20 | 116, 117 | ND490, |
| | | hematological and | | | | ND491 |
| | | neurological expressed 1 | | | | |
| 32570_at | L76465 | Hydroxyprostaglandin | HPGD | 21 | 118, 119 | ND528, |
| | | dehydrogenase 15-(NAD) | | | | ND529 |
| 37639_at | X07732 | hepsin (transmembrane | HPN | 22 | 120, 121 | ND595, |
| | | protease, serine 1) | | | | ND596 |
| 63673_at | AI635057 | HSBP1-Heat shock | HSBP1 | 23 | 122, 123 | ND702, |
| | | protein 27A | | | | 703 |
| 1232_s_at | M74587 | Insulin like growth | IGFBP1 | 24 | 124, 125 | ND608, |
| | | factor binding protein 1 | | | | 609 |
| | | precursor | | | | |
| 1804_at | X07730 | kallikrein-related | KLK3 | 25 | 126, 127, | ND438, |
| | | peptidase 3 | | | 128, 129 | ND439, |
| | | | | | | ND470, |
| | | | | | | ND471 |
| 217_at, | S39329 | kallikrein-related | KLK2 | 26 | 130, 131 | ND418, |
| 41721_at | | peptidase 2 | | | | ND419 |
| 62175_at | AI50156 | Homo sapiens laminin, | LAMA1 | 27 | 132, 133 | ND662, |
| | | alpha 1 | | | | ND663 |
| 60019_at, | AA947309.1 | Leucine rich repeat | LRRN1 | 28 | 134, 135 | ND428, |
| 56912_at | | neuronal 1-Homo | | | | ND429 |
| | | sapiens leucine-rich | | | | |
| | | repeats and calponin | | | | |
| | | homology (CH) domain | | | | |
| | | containing 4 (LRCH4) | | | | |
| 1083_s_at, | M35093 | Mucinl cell surface | MUC1 | 29 | 136, 137 | CH284, |
| 927_at | | associated protein | | | | CH285 |
| 52116_at | AI697679 | Myelin expression factor 2 | MYEF2 | 30 | 138, 139 | ND396, |
| | | | | | | ND397 |
| 35024_at | L37362 | OPRK1 receptor | OPRK1 | 31 | 140, 141 | ND404, |
| | | | | | | ND405 |
| 59233_at | W27060 | Homo sapiens SET | SETMAR | 32 | 142, 143 | ND492, |
| | | domain and mariner | | | | ND493 |
| | | transposase fusion gene | | | | |
| | | (SETMAR) transcript | | | | |
| | | variant 3, non-coding | | | | |
| | | RNA | | | | |
| — | — | Homo sapiens | LOC100506 | 33 | 144, 145 | ND488, |
| | | uncharacterized | 990 | | | ND489 |
| | | LOC100506990, | | | | |
| | | transcript variant 2 non- | | | | |
| | | coding RNA | | | | |
| 51776_s_at, | AI749525, | PDZK1 interacting | PDZK1IP1 | 34 | 146, 147 | ND500, |
| 31610_at, | U21049, | protein 1 | | | | ND501 |
| 59794_g_at | AA872415 | | | | | |
| 41281_s_at | AF060502 | Peroxisomal biogenesis | PEX10 | 35 | 148, 149 | CH139, |
| | | factor 10 | | | | CH140 |
| 40116_at | X16911 | Homo sapiens | PFKL | 36 | 150, 151 | ND708, |
| | | phosphofructokinase, | | | | ND709 |
| | | liver (PFKL) | | | | |
| 39175_at | D25328 | Homo sapiens | PFKP | 37 | 152, 153 | ND696, |
| | | phosphofructokinase, | | | | ND697 |
| | | platelet (PFKP) gene | | | | |
| 41094_at | Y10179 | Prolactin Induced | PIP | 38 | 154, 155 | ND502, |
| | | Protein | | | | ND503, |
| | | | | | | CH268, |
| | | | | | | CH269 |
| 37068_at | U24577 | phospholipase A2, group | PLA2G7 | 39 | 156, 157 | CH212, |
| | | VII (platelet-activating | | | | CH213 |
| | | factor acetylhydrolase, | | | | |
| | | plasma) | | | | |
| 63958_at | AI583077 | prostate stem cell | PSCA | 40 | 158, 159 | ND380, |
| | | antigen | | | | ND381 |
| 1739_at, | M99487 | Prostate-specific | PSMA | 41 | 160, 161 | ND402, |
| 1740_g_at | | membrane antigen | | | | ND403 |
| 33272_at | AA829286 | Serum amyloid A2 | SAA2 | 42 | 162, 163 | CH320, |
| | | | | | | CH321 |
| 36781_at | X01683 | Serpin peptidase | SERPINA1 | 43 | 164, 165 | ND446, |
| | | inhibitor Glade A | | | | ND447 |
| 54293_at | N30034 | Solute carrier family 10, | SLC10A7 | 44 | 166, 167 | ND734, |
| | | member 7 | | | | ND735 |
| 39926_at | U59913 | Homo sapiens SMAD | SMAD5 | 45 | 168, 169 | ND710, |
| | | family member 5 | | | | ND711 |
| | | (SMAD5) | | | | |
| 52576_s_at | AW007426 | Spondin 2 extracellular | SPON2 | 46 | 170, 171 | ND358, |
| | | matrix protein | | | | ND359 |
| 34342_s_at | AF052124 | Osteopontin:secreted | SPP1 | 47 | 172, 173 | ND472, |
| | | phophoprotein | | | | ND473 |
| 1938_at | K03218 | Homo sapiens v-src | SRC | 48 | 174, 175 | ND704, |
| | | sarcoma (Schmidt- | | | | ND705 |
| | | Ruppin A-2) viral | | | | |
| | | oncogene homolog | | | | |
| — | — | Homo sapiens tudor | TDRD1 | 49 | 176, 177 | ND726, |
| | | domain containing 1 | | | | ND727 |
| | | (TDRD1) | | | | |
| 32154_at | M36711 | transcription factor AP-2 | TFAP2A | 50 | 178, 179 | ND494, |
| | | alpha (activating | | | | ND495 |
| | | enhancer binding protein | | | | |
| | | 2 alpha) | | | | |
| 47890_at | AI921465 | Homo sapiens | TMC5 | 51 | 180, 181 | ND670, |
| | | transmembrane channel- | | | | 671 |
| | | like 5 (TMC5) | | | | |
| 45574_g_at | AA534688 | TPX2-microtubule | TPX2 | 52 | 182, 183 | ND436, |
| | | associated | | | | ND437 |
| 57239_at | AI439109 | Homo sapiens isolate | TRIB1 | 53 | 184, 185 | ND718, |
| | | TRIB1-VI-T tribbles- | | | | 719 |
| | | like protein 1 | | | | |
| 56508_at | W22687 | Tetraspanin 13 | TSPAN13 | 54 | 186, 187 | ND386, |
| | | | | | | ND387 |
| 6315 _f at | T50788 | UDP | UGT2B15 | 55 | 188, 189 | ND452, |
| | | glucuronosyltransferase | | | | ND453 |
| | | 2 family polypeptide | | | | |
| | | B15 | | | | |
| 33279_at | X80062 | acyl-CoA synthetase | ACSM3 | 235 | 293, 294 | ND632, |
| | | medium-chain family | | | | ND633 |
| | | member 3 | | | | |
| 39314_at | X77533 | Actin A Receptor, type | ACVR2B | 236 | — | |
| | | IIB | | | | |
| 41706_at | AJ130733 | alpha-methylacyl-CoA | AMACR | 237 | 352, 353, | ND800, |
| | | racemase | | | 354, 355 | ND801, |
| | | | | | | ND802, |
| | | | | | | ND803 |
| 35084_at | AC005263 | Anti-Mullerian hormone | AMH | 238 | — | |
| 36106_at | X01388 | Apolipoprotein C-III | ApoCIII | 239 | — | |
| 31355_at | U77629.1 | Achaete-scute complex | ASCL2 | 240 | — | |
| | | homolog 2 | | | | |
| 46188_at | AI422243 | Chromosome 7 open | C7orf68 | 242 | — | |
| | | reading frame 68 | | | | |
| 61650_at | AI820748 | Chromosome 1 open | C1orf64 | 241 | 295, 296 | ND712, |
| | | reading frame 64 | | | | ND713 |
| 37605_at | L10347 | Collagen, type II, alpha 1 | COL2A1 | 243 | — | |
| 39925_at | M95610 | collagen, type IX, alpha 2 | COL9A2 | 244 | — | |
| 40162_s_at | AC003107 | Cartilage Oligomeric | COMP | 245 | — | |
| | | Matrix protein precursor | | | | |
| 45399_at | T77033 | Cysteine-rich secretory | CRISPLD1 | 246 | 297, 298 | ND634, |
| | | protein LCCL domain | | | | ND635 |
| | | containing 1 | | | | |
| 37020_at | X56692 | C-reactive protein | CRP | 247 | — | |
| 35506_s_at | J03870 | Cystatin S | CST4 | 248 | 299, 300 | ND390, |
| | | | | | | ND391 |
| 34623_at | M97925 | Defensin alpha 5, Paneth | DEFAS | 249 | — | |
| | | cell specific | | | | |
| 52138_at | AI351043, | v-ets erythroblastosis | ERG | 250 | 356, 357, | ND832, |
| | AI351043 | virus E26 oncogene like | | | 358, 359 | ND833, |
| | | (avian) | | | | ND834, |
| | | | | | | ND835 |
| 45394_s_at | AA563933 | Family with sequence | FAM3D | 251 | 301-304 | ND510, |
| | | similarity 3, member D | | | | ND511, |
| | | | | | | CH238, |
| | | | | | | CH239 |
| 31685_at | Y08976 | FEV (ETS oncogene | FEV | 252 | — | |
| | | family) | | | | |
| 35905_s_at | U34995 | Glyceraldehyde-3- | GAPDH | 253 | — | |
| | | phosphate | | | | |
| | | dehydrogenase | | | | |
| 34235_at | AB018301 | Homo sapiens G-protein | GPR116 | 254 | 305, 306 | ND660, |
| | | coupled receptor | | | | ND661 |
| | | GPR116 | | | | |
| 32430_at | M73481 | Gastrin releasing peptide | GRPR | 255 | — | |
| | | receptor | | | | |
| 40327_at | U57052 | homeo box B13 | HOXB13 | 256 | — | |
| 36227_at | AF043129 | Interleukin 7 receptor | IL7R | 257 | | |
| 46958_at | AI868421 | Potassium voltage gated | KCNC2 | 258 | — | |
| | | channel, Shaw-related | | | | |
| | | subfamily, member 2 | | | | |
| 33606_g_at | AF019415 | NK2 homeobox | NKX2-2 | 259 | — | |
| 60501_s_at | AA573803 | Homo sapiens OUT | OTUD5 | 260 | — | |
| | | domain containing 5 | | | | |
| | | (OTUD5) | | | | |
| — | — | Homo sapiens prostate | PCA3 | 261 | 307, 308 | ND171, |
| | | cancer antigen 3 | | | | ND174 |
| | | NR_0153432.1 | | | | |
| 33703_f_at, | L05144 | Phophoenol pyruvate | PCK1 | 262 | — | |
| 33702 _f_ at | | carboxy kinase I | | | | |
| 39696_at | AB028974 | Paternally expressed 10 | PEG10 | 263 | — | |
| 58941 _at | AI765967 | Phospholipase A1 | PLAZA | 264 | — | |
| 62240_at | AI096692 | Proline rich 16 | PRR16 | 265 | — | |
| 33259_at | M81652 | Semenogelin II | SEMG2 | 266 | 309, 310 | ND474, |
| | | | | | | ND475 |
| 928_at | L02785 | Solute carrier 26, | SLC26A3 | 267 | — | |
| | | member 3 | | | | |
| 51847_at | AA001450 | Solute carrier family 44, | SLC44A5 | 268 | 311, 312 | ND360, |
| | | member 5 | | | | ND361 |
| 35716_at | AB008164 | Sulfotransferase | SULT1C2 | 269 | 313, 314 | ND476, |
| | | | | | | ND477 |
| 37898_r_at | AI985964 | Trefoil factor 3 | TFF3 | 270 | — | |
| 40328_at | X99268 | TWIST homolog 1 | TWIST1 | 271 | — | |
| 1651_at | U73379 | Ubiquitin-conjugating | UBE2C | 272 | — | |
| | | enzyme E2C | | | | |
| 44403_at | AI873501 | Clone HH0011_E05 | | 273 | — | |
| | | mRNA sequence | | | | |
| 40375_at | X63741.1 | Early growth response 3 | EGR3 | 327 | 360, 361 | ND676, |
| | | | | | | ND677 |
| 34936_at | AB012130 | Solute carrier family 4, | SLC4A7 | 328 | 362, 363 | ND666, |
| | | sodium biocarbonate co- | | | | ND667 |
| | | transporter, member 7 | | | | |
| 38473_at | M63180.1 | Threonyl-tRNA | TARS | 329 | 364, 365 | ND668, |
| | | synthetase | | | | ND669 |
| 43102_g_at | N93788.1, | Vacuolar protein sorting | VPS13B | 330 | 366, 367 | ND672, |
| | AI138355.1 | 13 homolog B | | | | ND673 |
| 60814_at | H65645.1 | Aldo-keto reductase | AKR1C1 | 331 | 368, 369 | ND820, |
| | | family 1, member C1 | | | | ND821 |
| 35482_at | M33375.1 | Aldo-keto reductase | AKR1C4 | 332 | 370, 371 | ND838, |
| | | family 1, member C4 | | | | ND839 |
| 40690_at | X54942.1 | CDC28 protein kinase | CKS2 | 333 | 372, 373 | ND812, |
| | | regulatory subunit 2 | | | | ND813 |
| 37305_at | U61145 | Enhancer of zeste | EZH2 | 334 | 374, 375 | ND818, |
| | | homolog 2 | | | | ND819 |
| 31859_at | J05070 | Matrix metallopeptidase 9 | MMP9 | 335 | 376, 377 | ND814, |
| | | | | | | ND815 |
| 50099_at | AI733116.1 | Membrane spanning 4- | MS4A8 | 336 | 378, 379 | ND664, |
| | | domains, subfamily A | | | | ND665 |
| | | member 88 | | | | |
| 575_s_at | M93036.1 | Epithelial cell adhesion | EPCAM | 337 | 380, 381 | ND898, |
| | | molecule | | | | ND899 |
| — | HQ605084.1 | PCAT1 long non-coding | PCAT1 | 418 | 414, 415 | ND904, |
| | | RNA | | | | ND905 |
| — | HQ605085.1 | PCAT14 long non-coding | PCAT14 | 419 | 416, 417 | ND906, |
| | | RNA | | | | ND907 |

TABLE 2
RNA Biomarkers Showing Reduced Expression Levels in Prostate Cancer Patients

| | | | | SEQ | PRIMER | |
|---|---|---|---|---|---|---|
| | GENBANK | | GENE | ID | SEQ ID | PRIMER |
| REPORTER | ACCESSION | GENE DESCRIPTION | SYMBOL | NO: | NOs | IDs |
| 32200_at | M24902 | acid phosphatase, | ACPP | 56 | 190, 191 | ND496, |
| | | prostate | | | | ND497 |
| 35834_at | X59766 | Alpha-2-glycoprotein 1, | AZGP1 | 57 | 192, 193 | CH161, |
| | | zinc-binding | | | | CH162 |
| 36780_at | M25915 | Clusterin | CLU | 58 | 194, 195 | ND698, |
| | | | | | | ND699 |
| 38700_at | M33146 | Cysteine and glycine- | CSRP1 | 59 | 196, 197, | DR583, |
| | | rich protein 1 | | | 198, 199 | DR584, |
| | | | | | | ND690, |
| | | | | | | ND691 |
| 65988_at | W19285 | Early b-cell factor 3 | EBF3 | 60 | 200, 201 | ND730, |
| | | | | | | ND731 |
| 38422_s_at | U29332 | 4.5 LIM domains | FHL2 | 61 | 202, 203 | DR569, |
| | | | | | | DR570 |
| 32749_s_at | AL050396 | filamin A | FLNA | 62 | 204, 205 | ND624, |
| | | | | | | ND625 |
| 53270_s_at | AW021867 | Homo sapiens mitogen- | MAP3K7 | 63 | 206, 207 | ND682, |
| | | activated protein kinase | | | | ND683 |
| | | kinase kinase 7 | | | | |
| 32149_at | AA532495 | microseminoprotein, | MSMB | 64 | 208, 209 | CH143, |
| | | beta- | | | | CH144 |
| 32847_at | U48959 | Myosin kinase | MYLK | 65 | 210, 211 | DR567, |
| | | | | | | DR568 |
| 33505_at, | AI887421, | Retinoic acid responder | RARRES1 | 66 | 212, 213 | DR575, |
| 1042_at, | U27185, | | | | | DR576 |
| 62940_f_at | AI669229 | | | | | |
| 64449_at | AI810399 | Selenoprotein M | SELM1 | 67 | 214, 215 | DR559, |
| | | | | | | DR560 |
| 32521_at | AF056087 | Secreted frizzled-related | SFRP1 | 68 | 216, 217 | DR555, |
| | | protein 1 | | | | DR556 |
| 39544_at | AB002351 | Synemin | SYNM | 69 | 218, 219 | DR579, |
| | | | | | | DR580 |
| 48039_at | AI634580 | Synaptopodin 2 | SYNPO2 | 70 | 220, 221 | DR737, |
| | | | | | | 738 |
| 3231_g_at | M75165 | Tropomyosin 2 | TPM2 | 71 | 222, 223 | DR565, |
| | | | | | | DR566 |
| 32755_at | X13839 | Actin SM | ACTA2 | 274 | — | |
| 1197_at | D00654 | Actin gamma2 | ACTG2 | 275 | — | |
| 32527_at | AI381790 | Unknown | C10orf116 | 276 | 315, 316 | ND571, |
| | | | | | | ND572 |
| 34203_at | D17408 | Calponin 1, basic, | CNN1 | 277 | 317, 318 | ND553, |
| | | smooth muscle | | | | ND554 |
| 57241_at | AI928870 | Dystrobrevin binding | DBNDD2 | 278 | — | |
| | | protein 1 | | | | |
| 38183_at | U13219 | Forkhead box F1 | FOXF1 | 279 | 319, 320 | DR557, |
| | | | | | | DR558 |
| 33396_at | U12472 | glutathione S-transferase | GSTP1 | 280 | — | |
| | | P1 | | | | |
| 53796_at | AI819282 | Potassium channel | KCNMA1 | 281 | 321, 322 | DR577, |
| | | | | | | DR578 |
| 49502_i_at | AI379607 | Mutated in CRC | MCC | 282 | 323, 324 | DR573, |
| | | | | | | DR574 |
| 767_at, | AF001548, | Myosin, heavy chain 11, | MYH11 | 283, | — | |
| 37407_s_at, | AF013570, | smooth muscle | | 284 | | |
| 773_at, | D10667, | | | | | |
| 774_g_at, | X69292 | | | | | |
| 32582_at | | | | | | |
| 37576_at | U52969 | Purkinje cell protein 4 | PCP4 | 285 | — | |
| 63827_at | AI479999 | Solute carrier family 22, | | 286 | 325, 326 | DR626, |
| | | SLC22A17 | | | | DR627 |
| | | member 17 | | | | |
| 33596_at | AJ001454 | Sparc/osteonectin, cwcv | SPOCK3 | 287 | — | |
| | | and katal like domains | | | | |
| | | proteoglycan (testican) 3 | | | | |
| 33412_at | Z83844 | Lectin, galactoside | LGALS-1 | 338 | 382, 383 | ND694, |
| | | binding soluble 1 | | | | ND695 |
| 42985_r_at | AI493076.1 | Aldo-keto reductase | AKR1C2 | 339 | 384, 385 | ND836, |
| | | family 1, member C2 | | | | ND837 |
| 6442_s_at, | AA628405, | BOC cell adhesion | BOC | 340 | 386,387 | ND896, |
| 52999_at | AA126704 | associated, oncogene | | | | ND897 |
| | | regulated | | | | |

For tests measuring the changes in frequency of RNA expression levels, it is essential to ensure adequate standardization. For this reason we have analyzed the NCBI database to identify reporters with the least variation between gene expression profiles, as shown in Table 3 below, in prostate cancer and healthy donor tissues. These reporters form a robust set of RNA reference genes that can be used where appropriate in tests involving quantification of RNA expression, such as in the modified NGS technology described herein.

TABLE 3
Reporters with Least Variation between Gene Expression Profiles

| | | | | SEQ | PRIMER | |
|---|---|---|---|---|---|---|
| | | GENE | GENE | ID | SEQ ID | PRIMER |
| REPORTER | PROBE | SYMBOL | DESCRIPTION | NO: | NOS: | IDs |
| 35184_at | AB011118 | ZFC3H1 | zinc finger, C3H1- | 72 | 224, 225 | ND514, |
| | | | type containing | | | ND515 |
| | | | CCDC131 | | | |
| 31826_at | AB014574 | FKBP15 | FK506 binding | 73 | 226, 227 | ND468, |
| | | | protein 15, 133 kDa | | | ND469 |
| 39811_at | AA402538 | C19orf50 | chromosome 19 open | 74 | 228, 229, | CH035, |
| | | | reading frame 50 | | 230 | CH036, |
| | | | | | | ND505 |
| 33397_at | AL050383 | CDIPT | CDP-diacylglycerol-- | 75 | 231, 232 | CH103, |
| | | | inositol 3- | | | CH104 |
| | | | phosphatidyltransferase | | | |
| 36003_at | AJ005698 | PARN | poly(A)-specific | 288 | — | |
| | | | ribonuclease | | | |
| | | | (deadenylation | | | |
| | | | nuclease) | | | |
| 35337_at | AL050254 | FBXO7 | F-box protein 7 | 289 | — | |
| F39020_at | U82938 | SIVA | CD27-binding (Siva) | 290 | — | |
| | | | protein polymerase | | | |
| 36027_at | AA418779 | POLR2F | PDGFA associated | 291 | — | |
| | | | protein 1 | | | |
| 38703_at | AF005050 | DNPEP | Aspartyl | 292 | — | |
| | | | aminopeptidase | | | |
| 66134 _f at | X95404.1 | CFL1 | Cofilin 1 | 341 | 388, 389 | ND844, |
| | | | | | | ND845 |
| 34643_at | M58458.1 | RPS4X | Ribosomal protein | 342 | 390, 391 | ND842, |
| | | | S4, X-lined | | | ND843 |

Table 4 lists reporters sharing common regulatory pathways with biomarkers listed in Tables 1 and 2.

TABLE 4
Reporters chosen for specific pathways

| | | | | SEQ | PRIMER | |
|---|---|---|---|---|---|---|
| | | GENE | GENE | ID | SEQ ID | PRIMER |
| REPORTER | PROBE | SYMBOL | DESCRIPTION | NO: | NOS: | IDs |
| 1779_s_at | M16750.1 | PIM | pim-1 oncogene | 343 | 392, 393 | ND822, |
| | | | | | | ND823 |
| 38127_at | Z48199 | SDC1 | syndecan 1 | 344 | 394, 395 | ND826, |
| | | | | | | ND827 |
| 1941_at | U33761.1 | SKP2 | S-phase kinase | 345 | 396, 397 | ND828, |
| | | | associated protein 2 | | | ND829 |
| 1542_at | X04571.1 | EGF | Epidermal growth | 346 | 398, 399 | ND868, |
| | | | factor | | | ND869 |
| 37327_at, | X00588 | EGFR | Epidermal growth | 347 | 400, 401 | ND870, |
| 1537_at | | | factor receptor | | | ND871 |
| 267_at, 40139_at | U88966, | MTOR | Mechanistic target | 348 | 402, 403 | ND878, |
| | L34075 | | of rapamycin | | | ND879 |
| 58901_at + 4 | AW021543, | PTEN | Phosphatase and | 349 | 404, 405, | ND872, |
| others | W73720, | | tensin homolog | | 406, 407, | ND873, |
| | U92436 | | | | 408, 409 | ND874, |
| | | | | | | ND875, |
| | | | | | | ND876, |
| | | | | | | ND877 |
| 1939_at, | M22898, | TP53 | Tumor protein p53 | 350 | 410, 411 | ND880, |
| 1974_s_at, | X02469, S6666 | | | | | ND881 |
| 31618_at | | | | | | |
| 589_at | M32313.1 | SRD5A1 | Steriod 5 alpha | 351 | 412, 413 | ND824, |
| | | | reductase, alpha | | | ND825 |
| | | | polypeptide 1 | | | |

Primers for the production of an RNA biomarker-specific amplicon were created using a multi-step primer design strategy. Specific intron-spanning primers were created to amplify an amplicon of a specific size (89 bp-160 bp) for use in Next Generation Sequencing (NGS).

The primers were designed using Primer 3 (v. 0.4.0) software and were checked to ensure that certain criteria were met:
No more than three C's or G's in the last five base pairs;
No runs (more than three) of G's in either primers;
No, or limited, self-complementarity or hairpin formation;
Primer BLAST of the primer set hits the cognate RNA target of the expected size; and
Consulted AceView for varying transcripts etc.

In order to use these RNA specific amplicon primer sets for RNA biomarker amplicon sequencing (RBAS) as described herein, nucleotides incorporating sequencing primers were added to the 5′ end of the primers in the first round PCR as described in Table 5 below, and a second set of primers used for a second round of PCR were used to add further sequences containing an index and adapter sequence.

TABLE 5
Specification of the added sequence to
the RNA biomarker specific primer used
for the first round PCR for biomarker
specific amplicon generation

| 1st round PCR | |
|---|---|
| Sequence added to | ACGACGCTCTTCCGATCT |
| forward primer 5′ end | (SEQ ID NO: 233) |
| Sequence added to | CGTGTGCTCTTCCGATCT |
| reverse primer 5′ end | (SEQ ID NO: 234) |

All primers used in the studies described herein were designed by the inventors and supplied by Life Technologies or IDT.

To determine the specificity and efficiency of the amplification, the RNA biomarker specific primers were first validated by performing real time SYBR green PCR quantification from relevant samples. A five-fold dilution series was used to construct relative standard curves for each primer set to determine PCR efficiency.

The relative amount of the marker gene in each of the samples tested was determined by comparing the cycle threshold (Ct value: number of PCR cycles required for the SYBR green fluorescent signal to cross the threshold exceeding background level within the exponential growth phase of the amplification curve). A separate PCR run of 32 cycles with no melting curve was set up, so that the amplicons could be electrophoresed on a 2% gel, cleaned up, and sequenced with standard Sanger chemistry using an Applied Biosystems 3130XL DNA sequencer to confirm the target.

### Example 1

### RNA Biomarker Amplicon Sequencing (RBAS) from Human Prostatectomy Tissue

To generate the RNA biomarker specific amplicons and prepare the amplicon library for each sample analyzed, cDNA prepared from RNA extracted from tumor and adjacent prostate gland tissue samples of each test subject was used separately as a template for eighty-eight individual PCR reactions with specific primer sets (i.e. oligonucleotide primers specific for the RNA biomarkers of interest including targets and references). The cDNA was synthesized from total RNA extracted from FFPE prostatectomy tissue using random hexamer primers for the production of the first strand cDNA using the SuperScript® VILO™ cDNA Synthesis Kit (Life Technologies). Each PCR reaction was mixed, and a duplicate aliquot was taken from each PCR product to create a duplicated amplicon library for each tissue sample. The amplicon libraries were then cleaned up to remove excess PCR reagents using paramagnetic bead technology and assessed for primer contamination and quantified. The Illumina adapter and index sequences were added to each amplicon library individually with a limited cycle PCR. The post adapter addition amplicon libraries were cleaned up to remove excess PCR reagents using paramagnetic beads, assessed to confirm the absence of primer contamination, verified for correct amplification of products and quantified. The cleaned and quantified post adapter addition amplicon libraries were diluted to 4 nM concentration and the libraries to be sequenced in parallel (4 libraries per test subject) were pooled in equimolar concentration to create a sequencing pool. The eighty eight biomarkers were split into 2 panels consisting of 42 biomarkers and 4 references. For each panel, one sequencing pool consisting of the duplicated amplicon libraries from the tumor and corresponding adjacent gland FFPE samples was prepared and diluted to 2 nM ready for sequencing. The 2 nM sequencing pool was denatured and further diluted to 10 pM or lower if necessary (containing 1% pre-denatured PhiX spike), and loaded into the MiSeq™ V2 300 cycle PE kit cartridge or other kits supplied by Illumina for sequencing using the MiSeq™ or the HiSeq™ 2000/HiSeq™ 2500 system if desired. A 101 cycle (single-end with indexing) sequencing program run on the MiSeq™ generates up to 21 million reads, and up to 2.1 GB of data.

Initial quality assessment of the sequencing run and demultiplexing (assignment of sequence reads to the appropriate test sample from which the library was prepared) was performed using the software provided with the MiSeq™ platform. The quality assessment of the resulting fastq formatted sequence reads acquired for each of the libraries and the biomarkers expression profile for each sample was analyzed using a novel software application developed in-house called D'Cipher. The assignment of the sequence reads to each of the corresponding RNA biomarker reference sequences was performed with the alignment algorithm BWA-MEM using default parameters (Li and Durbin, Bioinformatics, 26:589-95, 2010). The quality of a sample library was assessed by looking at the FASTQC report, the level of unaligned reads and gapped reads present in the libraries, and whether or not reads were aligning to more than one place. Using the BWA-MEM alignment tool, D'Cipher compiled the number of sequence reads aligning to each of the RNA biomarkers represented in the sequenced amplicon libraries, to generate the raw read counts per amplicon from which the differential expression analysis was performed. Different methods can be used for the scaling of the raw read counts aiming to normalize the wide count distribution produced by NGS. In the following examples, the raw read count obtained for each amplicon was scaled (divided) by the geometric mean of the raw read counts of three reference amplicons in the corresponding library. The reference amplicons represent RNA populations known to have low level of variation in expression across different prostate cancer and healthy donor control tissues. The normalized counts for each amplicon obtained by the expression in log₂ of the scaled read count, represent the expression profile of the corresponding RNA biomarkers in the analyzed sample and are used for differential expression analysis. In the differential expression analysis, the fold change represents the difference in normalized counts of an amplicon between compared libraries.

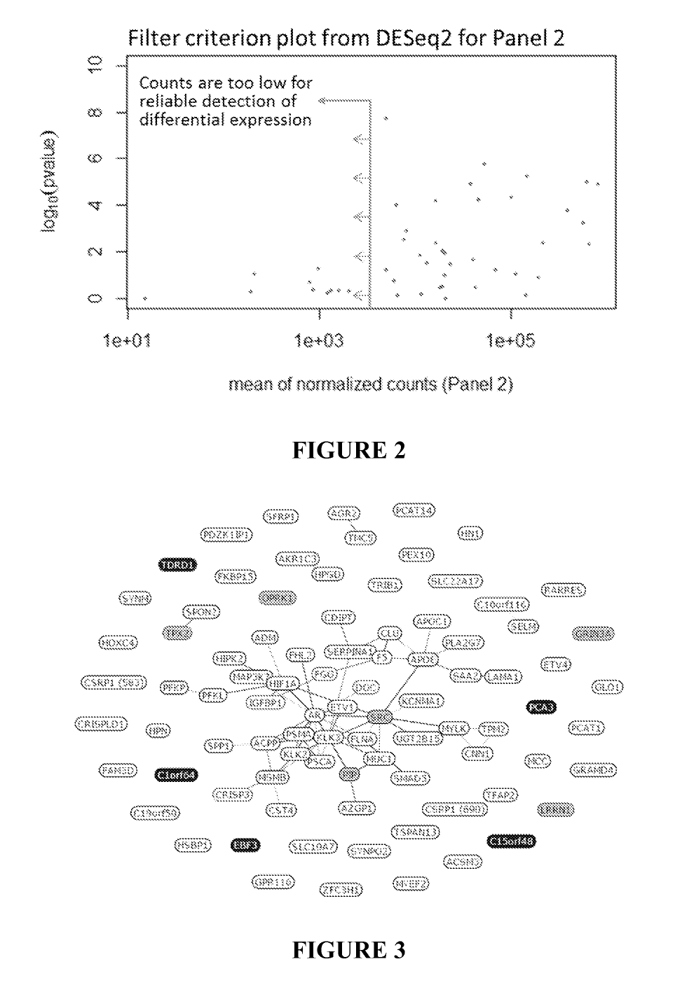

For weakly expressed genes, we have little to no chance of seeing differential expression, because the low read counts suffer from such high Poisson noise that any biological effect is drowned in the uncertainties from the read counting. To determine which biomarkers had too low counts to reliably test for differential expression, the independent filtering function implemented in the DESeq2 package (available from Bioconductor) was used (Love et al., bioRxiv preprint, 2014). The independent filtering function plots the filter criterion, which is the mean normalized count per biomarker across all samples over the −log₁₀ (p-value) calculated using DESeq2. This filter criterion allowed us to identify the overall lowest expressed genes across all samples that show no significant p-value (see FIG. 2). These genes were considered to have too low counts to reliably test for differential expression. In the tables of the following examples, these genes are indicated with an asterisk (*).

In the following examples an adjustment for contamination levels was performed. The average contamination level per target per library per RBAS run was obtained by calculating the average number of reads per library that align to screened biomarkers associated with library adapters that were not used in the present run or in the previous run, and dividing this average by the number of targets that were screened for in the RBAS run. All targets in the sample libraries that presented with counts below the average contamination per target per library for that run was considered ‘not detected’.

The differential expression profiles of an analyzed sample can be defined by the calculated fold changes or by whether or not the expression level is outside of a range deemed to be ‘normal’ and can be visualized onto an interaction network that enables the rapid identification of the specific pathways that were up- or down-regulated in the tested subject (see, for example, FIG. 3).

In the following examples, the analyzed subjects' samples were divided in five groups according to their respective assigned Gleason score: Group I consists of samples from one subject with a tumor with a Gleason score of 3+2; Group II consists of samples from eight subjects with tumors with a Gleason score of 3+3; Group III consists of samples from four subjects with tumors with a Gleason score of 3+4; Group IV consists of samples from four subjects with tumors with a Gleason score of 4+3; and Group V consists of samples from three subjects with tumors with a total Gleason score of 8, 9 or 10.

### Example 2

### Analysis of Prostate Cancer Tissue Samples Using RNA Biomarker Amplicon Sequencing and Comparison to their Own Glandular Tissue Sample Adjacent to the Cancerous Tissue

For this example, expression levels obtained in Example 1 through the RNA amplicon sequencing protocol were normalized using the reference genes and adjusted for contamination levels. Prostate cancer tissue samples of subjects from groups I, II, III, IV and V were compared to their own glandular tissue sample adjacent to the cancerous tissue. Biomarkers were selected based on their expression level in the tumor sample when the expression level of the biomarker in the tumor sample was more than 2.5 log₂ fold different from the expression of that biomarker in the adjacent glandular tissue. An example of the differential expression analysis of the tumor to its corresponding adjacent gland sample can be seen in FIG. 3, identifying biomarkers with altered frequency of expression in black for up-regulation and in grey for down-regulation.

Any biomarker showing a significant log₂ fold change in at least one of the samples was considered to have an altered frequency of expression. By selecting any biomarker that shows a significant difference in expression in at least one subject, we aim to capture the heterogeneity that is apparent in the progression of prostate cancer. In group I, five biomarkers were found to be up-regulated with an expression level that was more than 2.5 log₂ fold higher than the expression level in the adjacent glandular tissue and six biomarkers were found to be down-regulated compared to the adjacent glandular tissue. In group II, seventeen biomarkers were up-regulated and five were down-regulated. Twenty-four biomarkers were found to be both up- and down-regulated within the group. In group III, twenty-six biomarkers were up-regulated and eleven were down-regulated. Five biomarkers were both up- and down-regulated within this group. In group IV, thirty-seven biomarkers were up-regulated and eighteen were down-regulated. Four biomarkers were both up- and down-regulated within this group. This analysis was only conducted for two samples of group V because the adjacent glandular tissue sample for the subject with a tumor with a Gleason score of 4+5 was not available. For the two remaining subjects twelve biomarkers were identified to be up-regulated, seven were down-regulated. A list of these selected biomarkers is given in Tables 6A and B.

Tables 6A and B: Biomarkers with a Significant Difference in Expression in at Least One Subject when Comparing Tumor to its Adjacent Glandular Tissue

TABLE 6A

| T3 + 2 (n = 1) | | | T3 + 3 (n = 8) | | | T3 + 4 (n = 4) | | |
|---|---|---|---|---|---|---|---|---|
| | | UP/ | | | UP/ | | | UP/ |
| UP | DOWN | DOWN | UP | DOWN | DOWN | UP | DOWN | DOWN |
| FC > 2.5 | FC <− 2.5 | FC > \|2.5\| | FC > 2.5 | FC <− 2.5 | FC > \|2.5\| | FC > 2.5 | FC <− 2.5 | FC > \|2.5\| |
| C15orf48 | GRIN3A* | | ACSM3 | APOC1 | ADM* | ACSM3 | AZGP1 | ETV4* |
| C1orf64 | LRRN1* | | AZGP1 | ETV1 | AGR2 | APOC1 | EBF3* | GRIN3A* |
| EBF3* | OPRK1* | | CRISPLD1 | ETV4* | C15orf48 | APOE | HPGD | MUC1* |
| PCA3 | PIP* | | CST4 | HN1* | C1orf64 | C15orf48 | LAMA1* | PEX10 |
| TDRD1 | SRC | | F5 | RARRES | CRISP3 | C1orf64 | MSMB | TPX2* |
| | TPX2* | | HOXC4 | | DDC* | CRISP3 | OPRK1* | |
| | | | KLK3 | | EBF3* | CRISPLD1 | LOC100506990 | |
| | | | MSMB | | FAM3D | CST4 | PDZK1IP1 | |
| | | | MUC1* | | GPR116 | ETV1 | PIP* | |
| | | | SETMAR | | GRIN3A* | F5 | SAA2 | |
| | | | PDZK1IP1 | | HPN | FGG* | UGT2B15* | |
| | | | PSMA | | LAMA1* | GPR116 | | |
| | | | SAA2 | | LRRN1* | HOXC4 | | |
| | | | SERPINA1 | | MYEF2 | HPN | | |
| | | | SLC10A7 | | OPRK1* | IGFBP1** | | |
| | | | SPON2 | | PCA3 | LRRN1* | | |
| | | | TSPAN13 | | PIP* | PCA3 | | |
| | | | | | PLA2G7 | PLA2G7 | | |
| | | | | | PSCA | PSCA | | |
| | | | | | SFRP1 | PSMA | | |
| | | | | | TDRD1 | SLC10A7 | | |
| | | | | | TMC5 | SPON2 | | |
| | | | | | TPX2* | SPP1 | | |
| | | | | | UGT2B15* | SRC | | |
| | | | | | | TDRD1 | | |
| | | | | | | TMC5 | | |

TABLE 6B

| T4 + 3 (n = 4) | | | T4 + 4, 5 + 5 (n = 2) | | |
|---|---|---|---|---|---|
| | | UP/ | | | UP/ |
| UP | DOWN | DOWN | UP | DOWN | DOWN |
| FC > 2.5 | FC < −2.5 | FC > \|2.5\| | FC > 2.5 | FC < −2.5 | FC > \|2.5\| |

| | | | | |
|---|---|---|---|---|
| ACSM3 | C19orf50 | F5 | C19orf50 | ADM* |
| ADM* | CLU | FAM3D | GLO1 | GRIN3A* |
| AGR2 | CNN1 | SAA2 | HOXC4 | IGFBP1** |
| APOC1 | CSRP1.583 | UGT2B15* | HPGD | OPRK1* |
| C15orf48 | CSRP1.690 | | LAMA1* | LOC100506990 |
| C1orf64 | FLNA | | MYEF2 | PEX10 |
| CRISP3 | HN1* | | PSMA | SERPINA1 |
| CRISPLD1 | HSBP1 | | SPON2 | |
| CST4 | MCC | | SRC | |
| DDC* | MYLK | | TDRD1 | |
| FHL2 | LOC100506990 | | TPX2* | |
| GLO1 | PFKP | | UGT2B15* | |
| GPR116 | PIP* | | | |
| GRAMD4 | SELM | | | |
| GRIN3A* | SERPINA1 | | | |
| HOXC4 | SLC22A17 | | | |
| HPGD | SPON2 | | | |
| HPN | TPM2 | | | |
| LAMA1* | | | | |
| LRRN1* | | | | |
| MUC1* | | | | |
| MYEF2 | | | | |
| OPRK1* | | | | |
| PCA3 | | | | |
| PDZK1IP1 | | | | |
| PEX10 | | | | |
| PLA2G7 | | | | |
| PSCA | | | | |
| PSMA | | | | |
| SLC10A7 | | | | |
| SPP1 | | | | |
| SRC | | | | |
| TDRD1 | | | | |
| TMC5 | | | | |
| TPX2* | | | | |
| TRIB1 | | | | |
| TSPAN13 | | | | |

### Example 3

### Establishment of a Reference Standard and Comparison of Samples from Prostate Cancer Tissue to this Reference Standard Using RBAS

We designed a strategy to establish a reference standard based on non-cancerous glandular samples. The aim of a reference standard is to approximate the expression levels of the biomarkers in healthy prostate glands and their normal variation, in order to distinguish abnormal expression due to the formation of a prostate cancer tumor. Ideally, a reference standard (R) would be established with the expression levels of the biomarkers in a number of samples derived from ‘healthy’ prostate glands, as these would be representative of the normal expression levels of the biomarkers and their normal biological variation. However, it is very difficult to obtain healthy prostate glands since these are not removed when there is no clinical indication of disease. To obtain the closest approximation of a ‘Healthy’ reference, we established a reference standard based on the most ‘normal’ samples available to us.

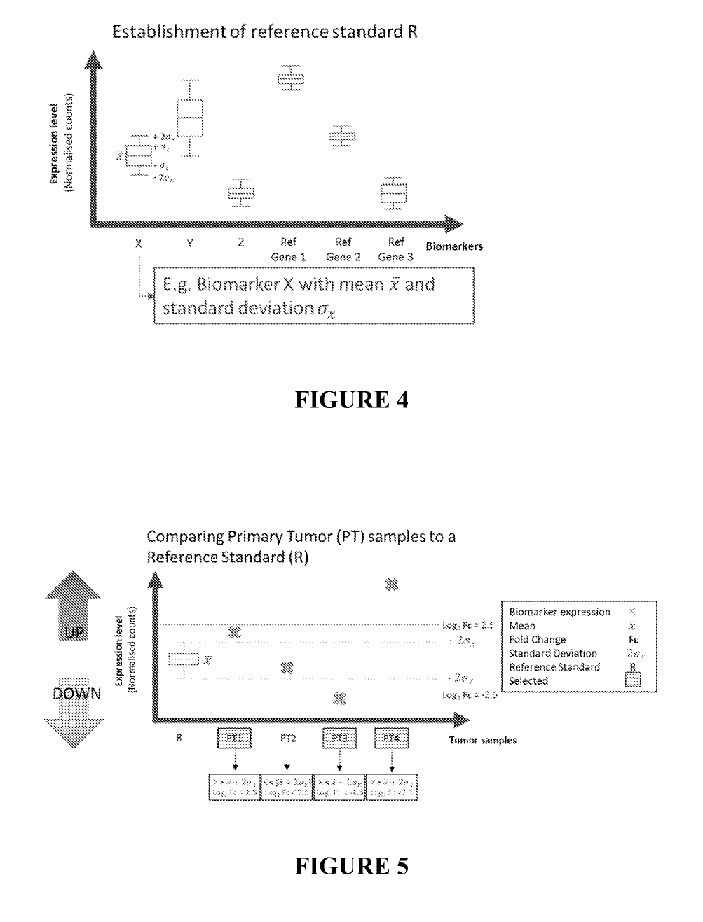

The most ‘normal’ samples available were samples from two subjects for whom a prostatectomy was indicated but upon histological examination no significant tumor was found. From one of these subjects, two independent samples were taken, so in total results from three samples were available. These samples served as a baseline ‘normal’ reference (N) and were used to construct reference standard version 1 (referred to as Rv1). Rv1 was established by calculating the mean of the expression levels per biomarker in the ‘normal’ samples, and the lower and upper ends of the ‘normal’ range were defined by the mean minus or plus two standard deviations respectively. FIG. 4 illustrates the theoretical mean ( x̅) expression levels of exemplified biomarkers x, y, z and their standard deviations (σₓ) used to establish the reference standard. When the expression of a biomarker in one or more normal samples was not detected, the lower end of the range was set to ‘not detected’. FIG. 5 illustrates the comparison of primary tumor (PT) samples to the reference standard (Rv1).

Next we compared the expression profile of the 88 biomarkers in the adjacent and tumor samples (T) from each subject of Example 2 to expression levels of Rv1. Biomarkers were determined to be differentially expressed in the tumor sample when they fulfilled at least one of the two following criteria:
1. The expression level of the biomarker in the tumor sample was more than 2.5 log₂ fold different from the mean expression of that biomarker in Rv1; and
2. The expression level of the biomarker in the tumor sample was outside of the ‘normal’ range of the expression level for that biomarker in Rv1.

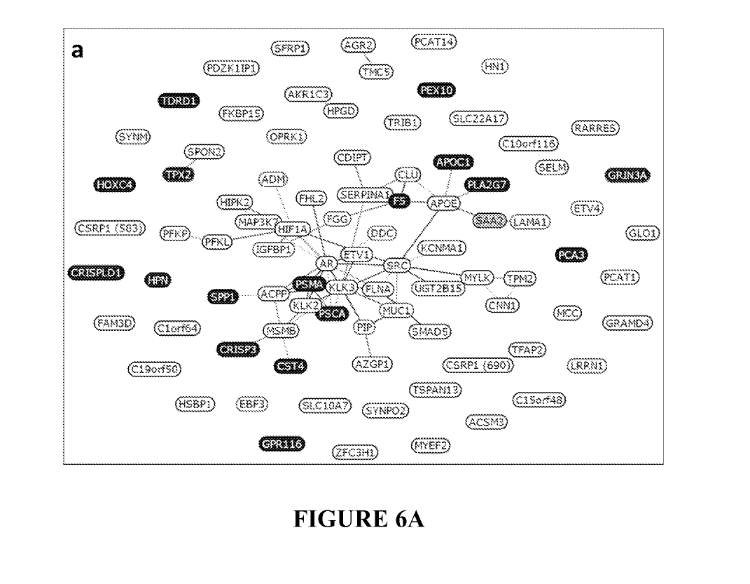

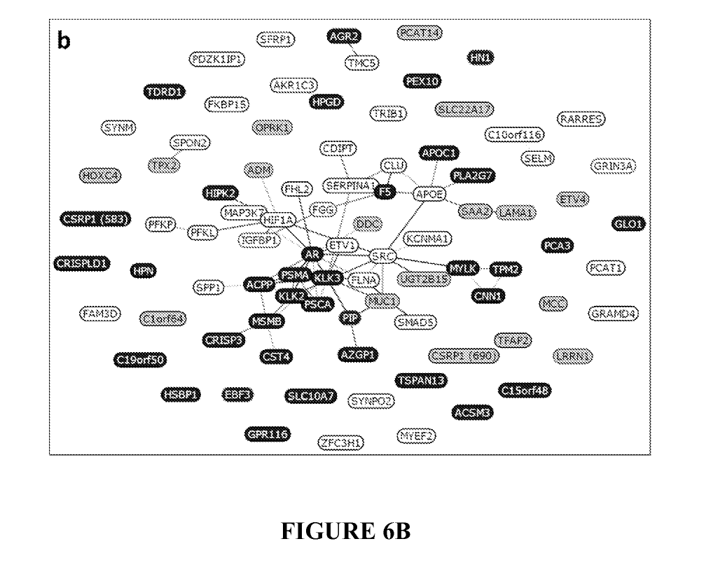

An example of the differential expression analysis of the subject's tumor tissue and its adjacent gland sample to the Rv1 is given in FIGS. 6A & 6B. Results for this comparison are based on chosen log₂ fold change thresholds of >2.5 log₂ fold change for up-regulation and <−2.5 log₂ fold change for down-regulation.

The result of this analysis shows differential expression in biomarkers in the comparison of the tumor versus Rv1 that was not identified in the comparison of the tumor versus the adjacent gland sample. Further investigation showed that, for at least some of these biomarkers, differential expression could be detected in the comparison of the adjacent gland to Rv1. This indicates that differential expression of that biomarker is already detectable in the adjacent gland sample due to possible field effect, and therefore was not detected when the tumor was compared to its own adjacent gland.

Consequently, the use of a reference standard minimizes the possible influence of a field effect when using a subject's own adjacent gland as a control sample and at the same time provides a range of biological variation for the investigated biomarkers. As such, this reference standard is employed as an alternative control sample to the adjacent non-cancerous glands of the subjects themselves.

To improve the estimation of the ‘normal’ biological range of expression of a biomarker, we explored the possibility of including other samples in the establishment of a reference standard. The next most ‘normal’ samples available were the adjacent glandular samples from subjects with low Gleason score tumors (3+2 and 3+3). One sample from a subject with a Gleason score of 3+2 and eight samples from subjects with a Gleason score of 3+3 were added to establish reference standard version 2 (Rv2). In these subjects, tumor development is likely to be limited and as such, would have the least field effect on the adjacent non-cancerous glandular tissue. Care was taken to use a sample that was taken from a section that was located in a different part of the prostate gland, again limiting the chance of incorporating field effect into the reference standard. Expression levels for each biomarker were checked for outliers by using the Grubbs' test (Grubbs, Technometrics, 11:1-21, 1969). A maximum of 1 value was removed when it proved to be outlying compared to the results of the other glands included in the reference standard establishment (p<0.05). In 18 of the 88 biomarkers, 1 value was removed because of its high chance of being an outlier. From then onwards, Rv2 was established in the same way as Rv1: the mean of the expression levels per biomarker was calculated for the samples included in the reference standard, and the lower and upper end of the ‘normal’ range were defined by the mean minus or plus two standard deviations, respectively. Differential expression was now only defined as an expression level of a biomarker detected in the tumor that is outside of the normal range of that biomarker in Rv2.

Next, the samples included in the reference standard were checked for the presence of field effect. This was done by comparing the expression levels of the biomarkers in the gland samples included in Rv2 to Rv1. Biomarkers that showed differential expression either by exceeding the threshold for fold change or by being outside of the normal range were indicated. Those biomarkers that are differentially expressed in the same direction as the differential expression detected in the corresponding tumor versus Rv1 comparison were then considered as being influenced by field effect. In 34 biomarkers, up to five samples of Rv2 presented with a field effect in those particular biomarkers.

In the establishment of reference standard version 3 (Rv3), this field effect was countered by removing those datapoints from the dataset before outlier removal, calculation of the mean and determination of the ‘normal’ biological range, which was done in the same way as for Rv2.

### Example 4

### Analysis of Group I and II Prostate Cancer Tissue Samples Using RBAS and Comparison to Rv2 and Rv3

For this example, expression levels obtained as described in Example 1 using the RBAS protocol from eight tumor samples from group II were compared to Rv2 and Rv3 established as per Example 3 above.

Any biomarker with an expression level outside of the normal biological range of the reference standard in at least one of the samples was considered as having an altered frequency of expression. By selecting any biomarker that shows a significant difference in expression in at least one subject, we aim to capture the heterogeneity that is apparent in the progression of prostate cancer. When comparing to Rv2, twenty-nine biomarkers were found to be up-regulated, seventeen biomarkers showed down-regulation in at least one of the (3+3) tumor samples, and thirteen biomarkers showed significant up-regulation in at least one and down-regulation in at least one other (3+3) sample. A list of these selected biomarkers is given in Table 7A. Table 7B lists the biomarkers selected when comparing the tumor samples of group II to Rv3. In this analysis, thirty biomarkers were found to be up-regulated, of which twenty-eight matched the ones up-regulated when comparing to Rv2; nineteen biomarkers were found to be down-regulated, of which seventeen matched the ones down-regulated when comparing to Rv2; and sixteen biomarkers showed significant up-regulation in at least one sample and down-regulation in at least one other sample, of which thirteen matched the ones selected when comparing to Rv2.

TABLE 7A
Up- and down-regulated biomarkers compared to
Rv2 in at least 1 subject from group I or II

| vs. Reference Std version 2 (Rv2) | | | vs. Reference Std Version 2 (Rv2) | | |
|---|---|---|---|---|---|
| Group I T3 + 2 (n = 1) | | | Group II T3 + 3 (n = 8) | | |
| | | UP/ | | | UP/ |
| | | DOWN | | | DOWN |
| UP | DOWN | x > x̅ + 2σx or | UP | DOWN | x > x̅ + 2σx or |
| x > x̅ + 2σx | x < x̅ − 2σx | x < x̅ − 2σx | x > x̅ + 2σx | x < x̅ − 2σx | x < x̅ − 2σx |

| | | | | |
|---|---|---|---|---|
| KCNMA1 | LRRN1* | ZFC3H1 | CDIPT | ADM* |
| | ACSM3 | ACPP | APOE | C15orf48 |
| | C10orf116 | ACSM3 | CLU | F5 |
| | F5 | AGR2 | CSRP1.583 | FLNA |
| | SLC22A17 | AKR1C3 | CSRP1.690 | GPR116 |
| | | AR.460 | ETV1 | HSBP1 |
| | | AZGP1 | ETV4* | KCNMA1 |
| | | C10orf116 | FAM3D | MCC |
| | | CRISP3 | FHL2 | MYEF2 |
| | | CRISPLD1 | HN1* | SAA2 |
| | | CST4 | LRRN1* | SELM |
| | | DDC* | MYLK | SLC22A17 |
| | | GLO1 | LOC100506990 | SYNPO2 |
| | | GRAMD4 | PEX10 | |
| | | HIF1A | PFKL | |
| | | HIPK2 | SRC | |
| | | HPGD | TPM2 | |
| | | IGFBP1* | | |
| | | KLK3 | | |
| | | PCA3 | | |
| | | PLA2G7 | | |
| | | PSMA | | |
| | | SERPINA1 | | |
| | | SFRP1 | | |
| | | SLC10A7 | | |
| | | SPON2 | | |
| | | TDRD1 | | |
| | | TMC5 | | |
| | | TRIB1 | | |

TABLE 7B
Up- and down-regulated biomarkers compared to
Rv3 in at least 1 subject from group I or II

| vs. Reference Std Version 3 (Rv3) | | | vs. Reference Std Version 3 (Rv3) | | |
|---|---|---|---|---|---|
| Group I T3 + 2 (n = 1) | | | Group II T3 + 3 (n = 8) | | |
| | | UP/ | | | UP/ |
| | | DOWN | | | DOWN |
| UP | DOWN | x > x̅ + 2σx or | UP | DOWN | x > x̅ + 2σx or |
| x > x̅ + 2σx | x < x̅ − 2σx | x < x̅ − 2σx | x > x̅ + 2σx | x < x̅ − 2σx | x < x̅ − 2σx |

| | | | | |
|---|---|---|---|---|
| KCNMA1 | LRRN1* | ZFC3H1 | CDIPT | ADM* |
| PCA3 | ACSM3 | ACPP | APOE | C15orf48 |
| TDRD1 | C10orf116 | ACSM3 | CLU | F5 |
| | F5 | AGR2 | CSRP1.583 | FLNA |
| | SLC22A17 | AKR1C3 | CSRP1.690 | GPR116 |
| | AR(460) | AZGP1 | ETV1 | HSBP1 |
| | TFAP2 | C10orf116 | ETV4* | KCNMA1 |
| | | CRISP3 | FAM3D | MCC |
| | | CRISPLD1 | FHL2 | MYEF2 |
| | | CST4 | HN1* | SAA2 |
| | | DDC* | LRRN1* | SELM |
| | | GLO1 | MYLK | SLC22A17 |
| | | GRAMD4 | LOC100506990 | SYNPO2 |
| | | HIF1A | PEX10 | APOC1 |
| | | HIPK2 | PFKL | AR.460 |
| | | HPGD | SRC | TFAP2 |
| | | IGFBP1* | TPM2 | |
| | | KLK3 | MUC1* | |
| | | PCA3 | OPRK1* | |
| | | PLA2G7 | | |
| | | PSMA | | |
| | | SERPINA1 | | |
| | | SFRP1 | | |
| | | SLC10A7 | | |
| | | SPON2 | | |
| | | TDRD1 | | |
| | | TMC5 | | |
| | | TRIB1 | | |
| | | HOXC4 | | |
| | | MSMB | | |

### Example 5

### Analysis of Group III (Gleason Scores of 3+4) Prostate Cancer Tissue Samples Using RBAS and Comparison to Rv2 and Rv3

Expression levels obtained through the RBAS protocol described in Example 1 from four tumor samples from subjects with a Gleason score of 3+4 were compared to Rv2 and Rv3, established as per Example 3. Biomarkers were selected based on their expression level in the tumor sample as per Example 4. A list of the selected biomarkers is given in Tables 8A and 8B below.

When comparing to Rv2, twenty-two biomarkers were found to be up-regulated and sixteen biomarkers were down-regulated. Six biomarkers presented with significant changes compared to Rv2 but showed both up- and down-regulation in two or more samples. When comparing to Rv3, twenty-five biomarkers were found to be up-regulated in at least one sample of group II, of which twenty-one matched those selected when comparing to Rv2. Seventeen biomarkers were found to be down-regulated, of which fifteen matched those selected when comparing to Rv2. Eight biomarkers were found to be both up- and down-regulated in two or more samples, of which seven matched those selected when comparing to Rv2.

TABLE 8A
Up- and Down-regulated biomarkers in at least 1 subject
with a tumor with a Gleason score of 3 + 4 compared to Rv2
vs. Reference Std Version 2 (Rv2)
Group III T3 + 4 (n = 4)

| | | UP/ |
|---|---|---|
| | | DOWN |
| UP | DOWN | x > x̅ + 2σx or |
| x > x̅ + 2σx | x < x̅ − 2σx | x < x̅ − 2σx |
| ACSM3 | C19orf50 | AR.460 |
| AGR2 | ADM* | F5 |
| AKR1C3 | CLU | GLO1 |
| AZGP1 | CSRP1.690 | HN1* |
| C15orf48 | CST4 | HSBP1 |
| CRISP3 | ETV4* | PEX10 |
| CRISPLD1 | HPGD | |
| DDC* | KCNMA1 | |
| FGG* | LOC100506990 | |
| GPR116 | PDZK1IP1 | |
| GRIN3A* | PFKL | |
| HIPK2 | SAA2 | |
| HPN | SELM | |
| IGFBP1* | SMAD5 | |
| PCA3 | SYNPO2 | |
| PLA2G7 | TFAP2 | |
| PSMA | | |
| SLC10A7 | | |
| SLC22A17 | | |
| SPON2 | | |
| TDRD1 | | |
| TRIB1 | | |

TABLE 8B
Up- and Down-regulated biomarkers in at least 1 subject
with a tumor with a Gleason score of 3 + 4 compared to Rv3
vs. Reference Std Version 3 (Rv3)
Group III T3 + 4 (n = 4)

| | | UP/ |
|---|---|---|
| | | DOWN |
| UP | DOWN | x > x̅ + 2σx or |
| x > x̅ + 2σx | x < x̅ − 2σx | x < x̅ − 2σx |
| ACSM3 | C19orf50 | AR.460 |
| AGR2 | ADM* | F5 |
| AKR1C3 | CLU | GLO1 |
| AZGP1 | CSRP1.690 | HN1* |
| C15orf48 | CST4 | HSBP1 |
| CRISP3 | ETV4* | PEX10 |
| CRISPLD1 | HPGD | SLC22A17 |
| DDC* | KCNMA1 | TFAP2 |
| FGG* | LOC100506990 | |
| GPR116 | PDZK1IP1 | |
| GRIN3A* | PFKL | |
| HIPK2 | SAA2 | |
| HPN | SELM | |
| IGFBP1* | SMAD5 | |
| PCA3 | SYNPO2 | |
| PLA2G7 | MUC1* | |
| PSMA | OPRK1* | |
| SLC10A7 | | |
| SPON2 | | |
| TDRD1 | | |
| TRIB1 | | |
| APOC1 | | |
| KLK3 | | |
| MYEF2 | | |
| PIP* | | |

### Example 6

### Analysis of Group IV (Gleason Score of 4+3) Prostate Cancer Tissue Samples Using RBAS and Comparison to Rv2 and Rv3

Expression levels obtained using the RBAS protocol in Example 1 from four tumor samples from subjects with a Gleason score of 4+3 were compared to Rv2 and Rv3, established as per Example 3. Biomarkers were selected based on their expression level in the tumor sample as per Example 4. A list of the selected biomarkers is given in Tables 9A and 9B below.

When comparing to Rv2, twenty-eight biomarkers were found to be up-regulated and twenty six biomarkers were down-regulated. Seven biomarkers presented with significant changes compared to Rv2 but showed both up- and down-regulation in two or more samples. When comparing to Rv3, twenty-nine biomarkers were found to be up-regulated in at least one sample of group II, of which twenty eight matched those selected when comparing to Rv2. Twenty-seven biomarkers were found to be down-regulated, of which twenty-six matched those selected when comparing to Rv2. Eight biomarkers were found to be both up- and down-regulated in two or more samples, of which seven matched those selected when comparing to Rv2.

TABLE 9A
Up- and Down-regulated biomarkers in at least 1 subject
with a tumor with a Gleason score of 4 + 3 compared to Rv2
vs. Reference Std Version 2 (Rv2)
Group IV T4 + 3 (n = 4)

| | | UP/ |
|---|---|---|
| | | DOWN |
| UP | DOWN | x > x̅ + 2σx or |
| x > x̅ + 2σx | x < x̅ − 2σx | x < x̅ − 2σx |
| ACSM3 | FKBP15 | C19orf50 |
| AGR2 | CDIPT | AR.460 |
| AKR1C3 | ADM* | AZGP1 |
| APOC1 | CLU | C10orf116 |
| C15orf48 | CNN1 | ETV4* |
| CRISP3 | CSRP1.583 | F5 |
| CRISPLD1 | CSRP1.690 | HN1* |
| CST4 | ETV1 | |
| DDC* | FLNA | |
| GLO1 | GRAMD4 | |
| GPR116 | HSBP1 | |
| GRIN3A* | KCNMA1 | |
| HIPK2 | LRRN1* | |
| HPGD | MAP3K7 | |
| HPN | MCC | |
| MYEF2 | MSMB | |
| PCA3 | MYLK | |
| PEX10 | LOC100506990 | |
| PLA2G7 | PDZK1IP1 | |
| PSMA | PFKP | |
| SLC10A7 | RARRES | |
| SPON2 | SELM | |
| SPP1 | SERPINA1 | |
| TDRD1 | SLC22A17 | |
| TFAP2 | SYNPO2 | |
| TMC5 | TPM2 | |
| TRIB1 | | |
| TSPAN13 | | |

TABLE 9B
Up- and Down-regulated biomarkers in at least 1 subject
with a tumor with a Gleason score of 4 + 3 compared to Rv3
vs. Reference Std Version 3 (Rv3)
Group IV T4 + 3 (n = 4)

| | | UP/ |
|---|---|---|
| | | DOWN |
| UP | DOWN | x > x̅ + 2σx or |
| x > x̅ + 2σx | x < x̅ − 2σx | x < x̅ − 2σx |
| ACSM3 | FKBP15 | C19orf50 |
| AGR2 | CDIPT | AR.460 |
| AKR1C3 | ADM* | AZGP1 |
| APOC1 | CLU | C10orf116 |
| C15orf48 | CNN1 | ETV4* |
| CRISP3 | CSRP1.583 | F5 |
| CRISPLD1 | CSRP1.690 | HN1* |
| CST4 | ETV1 | MUC1* |
| DDC* | FLNA | |
| GLO1 | GRAMD4 | |
| GPR116 | HSBP1 | |
| GRIN3A* | KCNMA1 | |
| HIPK2 | LRRN1* | |
| HPGD | MAP3K7 | |
| HPN | MCC | |
| MYEF2 | MSMB | |
| PCA3 | MYLK | |
| PEX10 | LOC100506990 | |
| PLA2G7 | PDZK1IP1 | |
| PSMA | PFKP | |
| SLC10A7 | RARRES | |
| SPON2 | SELM | |
| SPP1 | SERPINA1 | |
| TDRD1 | SLC22A17 | |
| TFAP2 | SYNPO2 | |
| TMC5 | TPM2 | |
| TRIB1 | OPRK1* | |
| TSPAN13 | | |
| HOXC4 | | |

### Example 7

### Analysis of Group V (Gleason Scores of 4+4, 4+5, 5+5) Prostate Cancer Tissue Samples Using RBAS and Comparison to Rv2 and Rv3

Expression levels obtained through the RBAS protocol in Example 1 from one tumor sample from a subject with a Gleason score of 4+4, one tumor sample from a subject with a Gleason score of 4+5 and one tumor sample from a subject with a Gleason score of 5+5 were compared to Rv2 and Rv3, established as per Example 3. Biomarkers were selected based on their expression level in the tumor sample as per Example 4. A list of the selected biomarkers is given in Tables 10A & 10B.

When comparing to Rv2, nineteen biomarkers were found to be up-regulated and twenty four biomarkers were down-regulated. Seven biomarkers presented with significant changes compared to Rv2 but showed both up- and down-regulation in two or more samples. When comparing to Rv3, twenty-two biomarkers were found to be up-regulated in at least one sample of group II, of which eighteen matched those selected when comparing to Rv2. Twenty-seven biomarkers were found to be down-regulated, of which twenty four matched those selected when comparing to Rv2. Eight biomarkers were found to be both up- and down-regulated in two or more samples, of which seven matched those selected when comparing to Rv2.

TABLE 10A
Up- and Down-regulated biomarkers in at least 1 subject with
a tumor with a Gleason score of 8, 9 or 10 compared to Rv2
vs. Reference Std Version 2 (Rv2)
Group V T4 + 4-->5 + 5 (n = 3)

| | | UP/ |
|---|---|---|
| | | DOWN |
| UP | DOWN | x > x̅ + 2σx or |
| x > x̅ + 2σx | x < x̅ − 2σx | x < x̅ − 2σx |
| ACSM3 | C10orf116 | ADM* |
| APOC1 | CLU | CST4 |
| APOE | CNN1 | ETV4* |
| AR.460 | CSRP1.583 | F5 |
| AZGP1 | CSRP1.690 | GLO1 |
| C1orf64 | FLNA | PEX10 |
| CRISP3 | GRAMD4 | PLA2G7 |
| CRISPLD1 | HSBP1 | |
| ETV1 | LRRN1* | |
| HN1* | MCC | |
| HPN | MYLK | |
| IGFBP1* | LOC100506990 | |
| MYEF2 | PDZK1IP1 | |
| PSMA | PFKL | |
| SLC10A7 | PSCA | |
| SPON2 | RARRES | |
| TDRD1 | SAA2 | |
| TMC5 | SELM | |
| TRIB1 | SERPINA1 | |
| | SLC22A17 | |
| | SMAD5 | |
| | SYNM | |
| | SYNPO2 | |
| | TPM2 | |

TABLE 10B
Up- and Down-regulated biomarkers in at least 1 subject with
a tumor with a Gleason score of 8, 9 or 10 compared to Rv3
vs. Reference Std Version 3 (Rv3)
Group V T4 + 4-->5 + 5 (n = 3)

| | | UP/ |
|---|---|---|
| | | DOWN |
| UP | DOWN | x > x̅ + 2σx or |
| x > x̅ + 2σx | x < x̅ − 2σx | x < x̅ − 2σx |
| ACSM3 | C10orf116 | ADM* |
| APOC1 | CLU | CST4 |
| APOE | CNN1 | ETV4* |
| AR.460 | CSRP1.583 | F5 |
| AZGP1 | CSRP1.690 | GLO1 |
| CRISP3 | FLNA | PEX10 |
| CRISPLD1 | GRAMD4 | PLA2G7 |
| ETV1 | HSBP1 | TFAP2 |
| HN1* | LRRN1* | |
| HPN | MCC | |
| IGFBP1* | MYLK | |
| MYEF2 | LOC100506990 | |
| PSMA | PDZK1IP1 | |
| SLC10A7 | PFKL | |
| SPON2 | PSCA | |
| TDRD1 | RARRES | |
| TMC5 | SAA2 | |
| TRIB1 | SELM | |
| DDC* | SERPINA1 | |
| HIPK2 | SLC22A17 | |
| PCA3 | SMAD5 | |
| UGT2B15* | SYNM | |
| | SYNPO2 | |
| | TPM2 | |
| | FHL2 | |
| | MUC1* | |
| | OPRK1* | |

### Example 8

### Comparison of Results Obtained by Comparing Prostate Cancer Tissue Samples to their Own Adjacent Glandular Sample, Rv1, Rv2 and Rv3

By comparing the results per subject across all methods used to select markers that are differentially expressed, we aimed to select markers that were differentially expressed no matter which reference was used to detect differential expression. The differential expression detected in these markers is considered to be the most reliable.

Tables 11A, B and C show examples of the comparison of the results of one sample from Group II, II and IV respectively across the four methods used.

TABLE 11A
Example of the comparison of the results for
one subject of Group II across all methods

| | T v A | T v Rv1 | T v Rv2 | T v Rv3 |
|---|---|---|---|---|
| Biomarker | S35T33 | S35T33 | S35T33 | S35T33 |
| F5 | + | + | + | + |
| GPR116 | + | + | + | + |
| PCA3 | + | + | + | + |
| PSMA | + | + | + | + |
| SLC10A7 | + | + | + | + |
| PLA2G7 | + | + | + | + |
| TDRD1 | + | + | + | + |
| ZFC3H1 | | + | + | + |
| CST4 | + | | + | + |
| MYEF2 | + | + | | + |
| TMC5 | | + | + | + |
| CRISP3 | + | | + | + |
| APOC1 | | + | | + |
| HIPK2 | | + | | + |
| HPN | + | + | | |
| TFAP2 | | + | | + |
| EBF3* | + | + | | |
| AGR2 | | + | | |
| FAM3D | + | | | |
| TSPAN13 | | + | | |
| C1orf64 | + | | | |
| HOXC4 | + | | | |
| LAMA1* | + | | | |
| PIP* | + | | | |
| TPX2* | + | | | |
| CDIPT | | − | − | − |
| CLU | | − | − | − |
| CSRP1 (690) | | − | − | − |
| ETV4* | | − | − | − |
| HSBP1 | | − | − | − |
| SELM | | − | − | − |
| AR (460) | | − | | − |
| FLNA | | | − | − |
| MCC | | | − | − |
| MYLK | | | − | − |
| LOC100506990 | | | − | − |
| SLC22A17 | | − | | − |
| TPM2 | | | − | − |
| C10orf116 | | − | | |
| FHL2 | | − | | |
| RARRES | − | | | |
| SMAD5 | | − | | |
| OPRK1* | + | | | − |
| LRRN1* | + | − | | |
| DDC* | ND | ND | ND | ND |
| FGG* | ND | ND | ND | ND |
| GRIN3A* | ND | ND | ND | ND |
| IGFBP1* | ND | ND | ND | ND |
| FKBP15 | | | | |
| C19ORF50 | | | | |
| ACPP | | | | |
| ACSM3 | | | | |
| ADM | | | | |

TABLE 11B
Example of the comparison of the results for
one subject of Group III across all methods

| | T v A | T v Rv1 | T v Rv2 | T v Rv3 |
|---|---|---|---|---|
| Biomarker | S41T34 | S41T34 | S41T34 | S41T34 |
| CRISPLD1 | + | + | + | + |
| SLC10A7 | + | + | + | + |
| TDRD1 | + | + | + | + |
| FGG* | + | + | + | + |
| APOC1 | + | + | | + |
| DDC* | | + | + | + |
| GPR116 | + | | + | + |
| PCA3 | + | + | | + |
| GRIN3A* | + | | + | + |
| AKR1C3 | | | + | + |
| C15orf48 | + | + | | |
| SLC22A17 | | | + | + |
| HPN | + | + | | |
| PIP* | | + | | |
| SPON2 | + | | | |
| SPP1 | + | | | |
| ACSM3 | + | | | |
| APOE | + | | | |
| C1orf64 | + | | | |
| CRISP3 | + | | | |
| ETV4* | + | | | |
| LRRN1* | + | | | |
| PLA2G7 | + | | | |
| PSCA | + | | | |
| PEX10 | − | − | − | − |
| HPGD | − | − | − | − |
| PDZK1IP1 | − | − | − | − |
| SAA2 | − | − | − | − |
| AR (460) | | − | − | − |
| CLU | | − | − | − |
| PFKL | | − | − | − |
| SMAD5 | | − | − | − |
| TFAP2 | | − | − | − |
| CSRP1 (690) | | − | | − |
| GLO1 | | | − | − |
| KCNMA1 | | | − | − |
| ZFC3H1 | | − | | |
| AZGP1 | − | | | |
| HIPK2 | | − | | |
| MAP3K7 | | − | | |
| MSMB | − | | | |
| SRC | | − | | |
| C19ORF50 | ND | − | − | − |
| CST4 | ND | − | − | − |
| ADM* | ND | − | − | − |
| F5 | ND | − | − | − |
| HN1* | ND | − | − | − |
| LOC100506990 | ND | − | − | − |
| ETV1 | + | − | | |
| MUC1* | ND | − | ND | − |
| EBF3* | − | − | ND | ND |
| HOXC4 | ND | ND | ND | ND |
| IGFBP1* | ND | ND | ND | ND |
| LAMA1* | ND | ND | ND | ND |
| OPRK1* | ND | ND | ND | ND |
| TPX2* | ND | ND | ND | ND |
| UGT2B15* | ND | ND | ND | ND |
| FKBP15 | | | | |
| CDIPT | | | | |
| ACPP | | | | |
| AGR2 | | | | |
| C10orf116 | | | | |

TABLE 11C
Example of the comparison of the results for
one subject of Group IV across all methods

| | T v A | T v Rv1 | T v Rv2 | T v Rv3 |
|---|---|---|---|---|
| Biomarker | S26T43 | S26T43 | S26T43 | S26T43 |
| C15orf48 | + | + | + | + |
| CRISP3 | + | + | + | + |
| CST4 | + | + | + | + |
| F5 | + | + | + | + |
| GPR116 | + | + | + | + |
| HPN | + | + | + | + |
| PCA3 | + | + | + | + |
| PEX10 | + | + | + | + |
| PLA2G7 | + | + | + | + |
| TDRD1 | + | + | + | + |
| AZGP1 | | + | + | + |
| GLO1 | | + | + | + |
| PSMA | | + | + | + |
| SLC10A7 | | + | + | + |
| DDC* | | | + | + |
| HIPK2 | | + | | + |
| MYEF2 | | + | | + |
| SPON2 | | | + | + |
| HOXC4 | + | | | + |
| ZFC3H1 | | + | | |
| AGR2 | | + | | |
| C1orf64 | + | | | |
| LAMA1* | + | | | |
| SETMAR | | + | | |
| TMC5 | | + | | |
| CDIPT | | − | − | − |
| CLU | | − | − | − |
| CSRP1 (690) | | − | − | − |
| ETV4* | | − | − | − |
| GRAMD4 | | − | − | − |
| HSBP1 | | − | − | − |
| SELM | | − | − | − |
| FLNA | | | − | − |
| RARRES | | | − | − |
| SLC22A17 | | − | | − |
| SYNPO2 | | | − | − |
| ETV1 | | − | | |
| FHL2 | | − | | |
| LRRN1* | | − | | |
| OPRK1* | | | | − |
| UGT2B15* | − | ND | ND | ND |
| FGG* | ND | ND | ND | ND |
| IGFBP1* | ND | ND | ND | ND |
| FKBP15 | | | | |
| C19ORF50 | | | | |
| ACPP | | | | |
| ACSM3 | | | | |

### Example 9

### Signature Predictions and Observations

(a) A Signature for Prostate Cancer Using Results from the Comparison Between Tumor Samples and their Own Adjacent Glandular Sample

Based on the results from Example 2, a combination of biomarkers was sought that was able to identify prostate cancer in groups II, III and IV. Groups I and V were not included due to low sample numbers. A combination of five biomarkers was identified, which included redundant biomarkers so that from these five, combinations of three biomarkers can be made that still identify all tumor samples as prostate cancer. The combinations and results are given in Table 12.

TABLE 12
Signature for prostate cancer formed by the comparison between tumor and adjacent glandular sample

| Biomarker | S32T33 | S2T33 | S9T33 | S19T33 | S20T33 | S21T33 | S24T33 | S35T33 | S33T34 |
|---|---|---|---|---|---|---|---|---|---|
| PCA3 | + | | + | + | + | + | | + | + |
| C1orf64 | + | | + | + | | + | + | + | + |
| TDR1 | + | + | | | + | + | | + | + |
| CST4 | | | | | + | + | | + | |
| PSMA | + | | + | + | + | + | | + | + |
| | Combination 1 | | | | | | | | |
| PCA3 | + | | + | + | + | + | | + | + |
| TDRD1 | + | + | | | + | + | | + | + |
| C1orf64 | + | | + | + | + | + | + | + | + |
| | Combination 2 | | | | | | | | |
| C1orf64 | + | | + | + | | + | + | + | + |
| TDRD1 | + | + | | | + | + | | + | + |
| CST4 | | | | | + | + | | + | |
| | Combination 3 | | | | | | | | |
| C1orf64 | + | | + | + | | + | + | + | + |
| TDRD1 | + | + | | | + | + | | + | + |
| PSMA | + | | + | + | + | + | | + | + |

| Biomarker | S34T34 | S37T34 | S41T34 | S38T43 | S39T43 | S26T43 | S40T43 |
|---|---|---|---|---|---|---|---|
| PCA3 | + | | + | + | + | + | + |
| C1orf64 | | + | + | | + | + | |
| TDR1 | + | + | + | | + | + | + |
| CST4 | + | | | + | + | + | + |
| PSMA | | | | + | | | + |
| | Combination 1 | | | | | | |
| PCA3 | + | | + | + | + | + | + |
| TDRD1 | + | + | + | | + | + | + |
| C1orf64 | | + | + | | + | + | |
| | Combination 2 | | | | | | |
| C1orf64 | | + | + | | + | + | |
| TDRD1 | + | + | + | | + | + | + |
| CST4 | + | | | + | + | + | + |
| | Combination 3 | | | | | | |
| C1orf64 | | + | + | | + | + | |
| TDRD1 | + | + | + | | + | + | + |
| PSMA | | | | + | | | + |

(b) A Signature for Prostate Cancer Using Biomarkers that are Up-Regulated in Every Method as Per Example 8

Based on the results of Example 8, a combination of biomarkers was sought that identified prostate cancer in all samples from groups II, III and IV no matter which reference was used to detect differential expression. A combination of nine biomarkers was identified in this way, using only those biomarkers that were consistently up-regulated with respect to the control. The combination and results are given in Tables 13A-C.

Tables 13A-C: Signature for Prostate Cancer Using Biomarkers that are Up-Regulated in Tumor Compared to all References (Adjacent (A), Rv1, Rv2 & Rv3)

TABLE 13A

| | ACSM3 | | | | ADM* | | | | AZGP1 |
|---|---|---|---|---|---|---|---|---|---|
| Subject | TvA | TvRv1 | TvRv2 | TvRv3 | TvA | TvRv1 | TvRv2 | TvRv3 | TvA |
| S02T33 | | | | | + | + | + | + | |
| S19T33 | | | | | | | | | |
| S20T33 | | + | + | + | | | | | + |
| S21T33 | | + | + | + | | | | | + |
| S24T33 | + | + | + | + | | | | | |
| S35T33 | | | | | | | | | |
| S32T33 | | + | | | + | | | | + |
| S9T33 | | | | | | | | | |
| S33T34 | | | | | | | | | |
| S34T34 | | + | + | + | | | | | |
| S37T34 | | | | | | | | | |
| S41T34 | + | | | | | | | | |
| S38T43 | + | | | | | | | | |
| S39T43 | + | + | + | + | + | | | | |
| S26T43 | | | | | | | | | |
| S40T43 | | + | + | + | | | | | |

| | AZGP1 | | | C15orf48 | | | |
|---|---|---|---|---|---|---|---|
| Subject | TvRv1 | TvRv2 | TvRv3 | TvA | TvRv1 | TvRv2 | TvRv3 |
| S02T33 | | | | | + | + | + |
| S19T33 | + | + | + | | | | |
| S20T33 | | | | + | + | | + |
| S21T33 | + | + | + | + | + | | + |
| S24T33 | | | | | | | |
| S35T33 | | | | | | | |
| S32T33 | + | + | + | + | | | |
| S9T33 | | | | | | | |
| S33T34 | | | | | | | |
| S34T34 | + | + | + | | + | | |
| S37T34 | + | + | + | + | + | + | + |
| S41T34 | | | | + | + | | |
| S38T43 | | | | | | | |
| S39T43 | | | | | | | |
| S26T43 | + | + | + | + | + | + | + |
| S40T43 | | | | | + | + | + |

TABLE 13B

| | CST4 | | | | KLK3 | | | | PLA2G7 |
|---|---|---|---|---|---|---|---|---|---|
| Subject | TvA | TvRv1 | TvRv2 | TvRv3 | TvA | TvRv1 | TvRv2 | TvRv3 | TvA |
| S02T33 | | | | | | | | | |
| S19T33 | | | | | | + | + | + | + |
| S20T33 | + | | | | + | + | | | + |
| S21T33 | + | | | | | + | | + | |
| S24T33 | | | | | | | | | |
| S35T33 | + | | + | + | | | | | + |
| S32T33 | | | | | | + | | | |
| S9T33 | | | | | | + | + | + | |
| S33T34 | | | | | | | | | + |
| S34T34 | + | | | | | + | | + | + |
| S37T34 | | | | | | + | | | |
| S41T34 | | | | | | | | | + |
| S38T43 | + | + | + | + | | | | | + |
| S39T43 | + | + | + | + | | | | | + |
| S26T43 | + | + | + | + | | | | | + |
| S40T43 | + | + | + | + | | | | | + |

| | PLA2G7 | | | SLC10A7 | | | |
|---|---|---|---|---|---|---|---|
| Subject | TvRv1 | TvRv2 | TvRv3 | TvA | TvRv1 | TvRv2 | TvRv3 |
| S02T33 | | | | | + | + | + |
| S19T33 | + | + | + | | + | | + |
| S20T33 | | + | + | | + | | |
| S21T33 | | | | | | | |
| S24T33 | | | | | + | | + |
| S35T33 | + | + | + | + | + | + | + |
| S32T33 | | | | | + | + | + |
| S9T33 | | | | | | | |
| S33T34 | + | + | + | | + | + | + |
| S34T34 | + | + | + | | + | + | + |
| S37T34 | | | | | + | | + |
| S41T34 | | | | + | + | + | + |
| S38T43 | + | + | + | | + | + | + |
| S39T43 | | + | + | + | + | + | + |
| S26T43 | + | + | + | | + | + | + |
| S40T43 | + | + | + | | + | + | + |

TABLE 13C

TMC5

| | | T v | T v | T v |
|---|---|---|---|---|
| Subject | T v A | Rv1 | Rv2 | Rv3 |
| S02T33 | | | | |
| S19T33 | + | | | |
| S20T33 | + | + | + | + |
| S21T33 | + | | | |
| S24T33 | | | | |
| S35T33 | | + | + | + |
| S32T33 | | | | |
| S9T33 | | | | |
| S33T34 | + | | | |
| S34T34 | | + | | |
| S37T34 | | | | |
| S41T34 | | | | |
| S38T43 | + | + | + | + |
| S39T43 | | | | |
| S26T43 | | + | | |
| S40T43 | | | | |

(c) A Signature for Prostate Cancer Using Biomarkers that are Up- or Down-Regulated in Every Method as Per Example 8

Based on the results of Example 8, we then sought to identify a combination of biomarkers that identified prostate cancer in all samples from groups II, III and IV no matter which reference was used to detect differential expression. A combination of seven biomarkers was identified in this way, using biomarkers that were consistently up- or down-regulated with respect to the control. The combination and results are given in Tables 14A and B.

Tables 14A and B: Signature for Prostate Cancer Using Biomarkers that are Up- or Down-Regulated in Tumor Compared to all References (Adjacent (A), Rv1, Rv2 & Rv3)

TABLE 14A

| | AZGP1 | | | | ADM* | | | | C15orf48 |
|---|---|---|---|---|---|---|---|---|---|
| Subject | TvA | TvRv1 | TvRv2 | TvRv3 | TvA | TvRv1 | TvRv2 | TvRv3 | TvA |
| S02T33 | | | | | + | + | + | + | |
| S19T33 | | + | + | + | − | − | − | − | − |
| S20T33 | + | | | | | | | | + |
| S21T33 | + | + | + | + | | | | | + |
| S24T33 | | | | | − | − | − | − | |
| S35T33 | | | | | | | | | |
| S32T33 | + | + | + | + | + | | | | + |
| S9T33 | | | | | | | | | |
| S33T34 | | | | | | | | | |
| S34T34 | | + | + | + | | | | | |
| S37T34 | | + | + | + | | | | | + |
| S41T34 | | | | | | − | − | − | + |
| S38T43 | | | | | | − | − | − | |
| S39T43 | | | | | + | | | | |
| S26T43 | | + | + | + | | | | | + |
| S40T43 | | | | | | | | | |

| | C15orf48 | | | ETV4* | | | |
|---|---|---|---|---|---|---|---|
| Subject | TvRv1 | TvRv2 | TvRv3 | TvA | TvRv1 | TvRv2 | TvRv3 |
| S02T33 | + | + | + | | − | − | − |
| S19T33 | − | − | − | − | − | − | − |
| S20T33 | + | | + | | − | | |
| S21T33 | + | | + | − | − | | |
| S24T33 | | | | | | | |
| S35T33 | | | | | − | − | − |
| S32T33 | | | | − | − | − | − |
| S9T33 | | | | | − | | − |
| S33T34 | | | | − | − | − | − |
| S34T34 | + | | | | − | − | − |
| S37T34 | + | + | + | | | | |
| S41T34 | + | | | + | | | |
| S38T43 | | | | | + | + | + |
| S39T43 | | | | | − | − | − |
| S26T43 | + | + | + | | − | − | − |
| S40T43 | + | + | + | | − | − | − |

TABLE 14B

| | GPR116 | | | | TDRD1 | | | | TMC5 | | | |
|---|---|---|---|---|---|---|---|---|---|---|---|---|
| Subject | TvA | TvRv1 | TvRv2 | TvRv3 | TvA | TvRv1 | TvRv2 | TvRv3 | TvA | TvRv1 | TvRv2 | TvRv3 |
| S02T33 | | | + | + | + | | | + | | | | |
| S19T33 | | | | | | | | | + | | | |
| S20T33 | | | | | + | + | | + | + | + | + | + |
| S21T33 | | | | | + | | | + | + | | | |
| S24T33 | | | | | − | | | | − | | | |
| S35T33 | + | + | + | + | + | + | + | + | | + | + | + |
| S32T33 | | | | | + | + | | + | | | | |
| S9T33 | − | − | − | − | − | | | | | | | |
| S33T34 | + | + | + | + | + | + | + | + | + | | | |
| S34T34 | | | | + | + | + | + | | + | | | |
| S37T34 | | | | | + | | | + | | | | |
| S41T34 | + | | + | + | + | + | + | + | | | | |
| S38T43 | | + | + | + | | | | | + | + | + | + |
| S39T43 | + | | | | + | + | + | + | | | | |
| S26T43 | + | + | + | + | + | + | + | + | | + | | |
| S40T43 | + | + | + | + | + | + | | + | | | | |

### Example 10

We reviewed each Gleason group independently and identified a signature that was common across the subjects of that group. For example, Group IV had eleven biomarkers that were consistently differentially expressed. This eleven biomarker signature was then compared to all other subjects to determine whether it was specific to Group IV.

We made the observation that subject 35 looked significantly different in these eleven biomarkers from other members of its Group (i.e. Group II) but aligned well with members of Group IV apart from one biomarker (PEX10). The combination of these observations is given in Tables 15A, parts I-III, and 15B, parts I-III.

This observation indicates that it may be possible to use molecular biomarker profiling to identify subgroups or re-categorize groups originally organized by Gleason score.

TABLE 15A
Signature common across the subjects of Group IV

| | CRISP3 | | | | CSRP1 (690) | | | | CST4 | | | | F5 | | | |
|---|---|---|---|---|---|---|---|---|---|---|---|---|---|---|---|---|
| | | | | Tv | | | | Tv | | | | Tv | | | | Tv |
| Subject | TvA | TvRv1 | TvRv2 | Rv3 | TvA | TvRv1 | TvRv2 | Rv3 | TvA | TvRv1 | TvRv2 | Rv3 | TvA | TvRv1 | TvRv2 | Rv3 |
| Group 2 | | | | | | | | | | | | | | | | |
| S02T33 | − | | | | | | | | | | | | | | | |
| S19T33 | + | | + | + | | − | − | − | | | | | | | | |
| S20T33 | | | | | | − | − | − | + | | | | | | | |
| S21T33 | + | | | | | − | − | − | + | | | | | − | | |
| S24T33 | − | | | | | | | | | | | | | | | |
| S35T33 | + | | + | + | | − | − | − | + | | + | + | + | + | + | + |
| S32T33 | + | | | | | − | | − | | | | | | − | − | − |
| S9T33 | | | | | | − | | − | | | | | | − | − | − |
| Group 3 | | | | | | | | | | | | | | | | |
| S33T34 | + | + | + | + | | − | | | | | | | + | + | + | + |
| S34T34 | | | | | | − | − | − | + | − | − | − | | + | + | + |
| S37T34 | | | | | | | | | | | | | | − | − | − |
| S41T34 | + | | | | | − | | − | | − | − | − | | − | − | − |
| Group 4 | | | | | | | | | | | | | | | | |
| S38T43 | + | | + | + | − | − | − | − | + | + | + | + | + | + | + | + |
| S39T43 | + | | | | | − | − | − | + | + | + | + | − | − | − | − |
| S26T43 | + | + | + | + | | − | − | − | + | + | + | + | + | + | + | + |
| S40T43 | + | + | + | + | | − | | − | + | + | + | + | + | + | + | + |

| GPR116 | | HPN | | PCA3 | | PEX10 | |
|---|---|---|---|---|---|---|---|
| | Tv | | Tv | | Tv | | Tv |

| | | | | | | | | | | | | | | | | |
|---|---|---|---|---|---|---|---|---|---|---|---|---|---|---|---|---|
| Subject | TvA | TvRv1 | TvRv2 | Rv3 | TvA | TvRv1 | TvRv2 | Rv3 | TvA | TvRv1 | TvRv2 | Rv3 | TvA | TvRv1 | TvRv2 | Rv3 |

II

| | | | | | | | | | | | | | | | | |
|---|---|---|---|---|---|---|---|---|---|---|---|---|---|---|---|---|
| Group 2 | | | | | | | | | | | | | | | | |
| S02T33 | | | + | + | | | | | | + | | + | | | | |
| S19T33 | | | | | + | + | | | + | + | | + | | | | |
| S20T33 | | | | | + | + | | | + | + | | + | | | | |
| S21T33 | | | | | + | + | | | + | + | | + | | − | | |
| S24T33 | | | | | − | | | | − | | | | | | | |
| S35T33 | + | + | + | + | + | + | | | + | + | + | + | | | | |
| S32T33 | | | | | + | + | | | + | + | | + | | | | |
| S9T33 | − | − | − | − | | | | | + | + | | + | | − | − | − |
| Group 3 | | | | | | | | | | | | | | | | |
| S33T34 | + | + | + | + | + | + | + | + | + | + | + | + | | + | + | + |
| S34T34 | | | | | + | + | + | + | + | + | | + | + | + | + | + |
| S37T34 | | | | | + | | | | | + | | + | | | | |
| S41T34 | + | | + | + | + | + | | | + | + | | + | − | − | − | − |
| Group 4 | | | | | | | | | | | | | | | | |
| S38T43 | | + | + | + | + | | | | + | + | | + | + | + | + | + |
| S39T43 | + | | | | + | | | | + | + | | + | | − | | |
| S26T43 | + | + | + | + | + | + | + | + | + | + | + | + | + | + | + | + |
| S40T43 | + | + | + | + | + | + | + | + | + | + | + | + | + | + | + | + |

| PLA2G7 | | SLC10A7 | | SLC22A17 | |
|---|---|---|---|---|---|
| | Tv | | Tv | | Tv |

| Subject | TvA | TvRv1 | TvRv2 | Rv3 | TvA | TvRv1 | TvRv2 | Rv3 | TvA | TvRv1 | TvRv2 | Rv3 |
|---|---|---|---|---|---|---|---|---|---|---|---|---|
| III | | | | | | | | | | | | |
| Group 2 | | | | | | | | | | | | |
| S02T33 | | | | | + | + | + | | | + | + | |
| S19T33 | + | + | + | + | | + | | + | | | | |
| S20T33 | + | | + | + | | + | | | | − | | |
| S21T33 | | | | | | | | | | − | − | − |
| S24T33 | − | | | | | + | | + | | − | | |
| S35T33 | + | + | + | + | + | + | + | + | | − | | − |
| S32T33 | | | | | | + | + | + | | − | | |
| S9T33 | | | | | | | | | | | | |
| Group 3 | | | | | | | | | | | | |
| S33T34 | + | + | + | + | | + | + | + | | − | | |
| S34T34 | + | + | + | + | | + | + | + | | − | | − |
| S37T34 | | | | | | + | | + | | − | | |
| S41T34 | + | | | | + | + | + | + | | | + | + |
| Group 4 | | | | | | | | | | | | |
| S38T43 | + | + | + | + | | + | + | + | | − | | − |
| S39T43 | + | | + | + | + | + | + | + | − | − | − | − |
| S26T43 | + | + | + | + | | + | + | + | | − | | − |
| S40T43 | + | + | + | + | | + | + | + | | − | − | − |

TABLE 15B
Comparison of Subject 35, from Group II with subjects from Group IV

| | | | | | | | | | | | | | F5 | | | |
|---|---|---|---|---|---|---|---|---|---|---|---|---|---|---|---|---|
| | CRISP3 | | | | CSRP1 (690) | | | | CST4 | | | | | | | Tv |
| Subject | TvA | TvRv1 | TvRv2 | TvRv3 | TvA | TvRv1 | TvRv2 | TvRv3 | TvA | TvRv1 | TvRv2 | TvRv3 | TvA | TvRv1 | TvRv2 | Rv3 |

I

| Group 4 | | | | | | | | | | | | | | | | |
|---|---|---|---|---|---|---|---|---|---|---|---|---|---|---|---|---|
| S38T43 | + | | + | + | − | − | − | − | + | + | + | + | + | + | + | + |
| S39T43 | + | | | | | − | − | − | + | + | + | + | − | − | − | − |
| S26T43 | + | + | + | + | | − | − | − | + | + | + | + | + | + | + | + |
| S40T43 | + | + | + | + | | − | | − | + | + | + | + | + | + | + | + |
| S35T33 | + | | + | + | | − | − | − | + | | + | + | + | + | + | + |
| | | | | | | | | | | | | | PEX10 | | | |
| | GPR116 | | | | HPN | | | | PCA3 | | | | | | | Tv |
| Subject | TvA | TvRv1 | TvRv2 | TvRv3 | TvA | TvRv1 | TvRv2 | TvRv3 | TvA | TvRv1 | TvRv2 | TvRv3 | TvA | TvRv1 | TvRv2 | Rv3 |

II

| Group 4 | | | | | | | | | | | | | | | | |
|---|---|---|---|---|---|---|---|---|---|---|---|---|---|---|---|---|
| S38T43 | | + | + | + | + | | | | + | + | | + | + | + | + | + |
| S39T43 | + | | | | + | | | | + | + | | + | | − | | |
| S26T43 | + | + | + | + | + | + | + | + | + | + | + | + | + | + | + | + |
| S40T43 | + | + | + | + | + | + | + | + | + | + | + | + | + | + | + | + |
| S35T33 | + | + | + | + | + | + | | | + | + | + | + | | | | |
| | PLA2G7 | | | | SLC10A7 | | | | SLC22A17 | | | | | | | |
| Subject | TvA | TvRv1 | TvRv2 | TvRv3 | TvA | TvRv1 | TvRv2 | TvRv3 | TvA | TvRv1 | TvRv2 | TvRv3 | | | | |
| III | | | | | | | | | | | | | | | | |
| Group 4 | | | | | | | | | | | | | | | | |
| S38T43 | + | + | + | + | | + | + | + | | − | | − | | | | |
| S39T43 | + | | + | + | + | + | + | + | − | − | − | − | | | | |
| S26T43 | + | + | + | + | | + | + | + | | − | | − | | | | |
| S40T43 | + | + | + | + | | + | + | + | | − | − | − | | | | |
| S35T33 | + | + | + | + | + | + | + | + | | − | | − | | | | |

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, method, method step or steps, for use in practicing the present invention. All such modifications are intended to be within the scope of the claims appended hereto.

Unless otherwise defined, all technical and scientific terms used herein have the same meaning as commonly understood by one of ordinary skill in the art to which this invention pertains. All of the publications, patent applications and patents cited in this application are herein incorporated by reference in their entirety to the same extent as if each individual publication, patent application or patent was specifically and individually indicated to be incorporated by reference in its entirety.

SEQ ID NO: 1-419 are set out in the attached Sequence Listing. The codes for nucleotide sequences used in the attached Sequence Listing, including the symbol “n,” conform to WIPO Standard ST.25 (1998), Appendix 2, Table 1.

1. A method for predicting a likelihood of the presence of prostate cancer in a test subject, comprising:
(a) measuring the expression levels of a plurality of RNA biomarkers in a biological sample obtained from the subject, wherein the plurality of RNA biomarkers comprises at least three RNA biomarkers selected from the group consisting of: RNA sequences corresponding to DNA sequences provided in SEQ ID NO: 1-75, 235-292, 327-351, 418 and 419;
(b) comparing the expression level of each of the plurality of RNA biomarkers in the biological sample with a predetermined reference standard for the RNA biomarker; and
(c) predicting the likelihood of the presence of prostate cancer in the subject based on a comparison of the expression level of each of the plurality of RNA biomarkers with the predetermined reference standard for the RNA biomarker.

2. The method of claim 1, wherein expression levels of the plurality of RNA biomarkers above or below the predetermined reference standards is indicative of the presence of prostate cancer in the subject.

3. The method of claim 1, wherein expression levels of the plurality of RNA biomarkers that are more than 2.5 log₂ fold higher or lower than the predetermined reference standards and/or are outside normal ranges of expression of the RNA biomarkers in the predetermined reference standards are indicative of the presence of prostate cancer in the subject.

4. The method of claim 1, wherein the at least three RNA biomarkers correspond to DNA sequences selected from the groups consisting of:
(a) SEQ ID NO: 41 (PSMA), SEQ ID NO: 49 (TDRD1), SEQ ID NO: 241 (Clorf64), SEQ ID NO: 248 (CST4), and SEQ ID NO: 261 (PCA3);
(b) SEQ ID NO: 1 (ADM), SEQ ID NO: 7 (C15orf48), SEQ ID NO: 25 (KLK3), SEQ ID NO: 39 (PLA2G7), SEQ ID NO: 44 (SLC10A7), SEQ ID NO: 51 (TMC5), SEQ ID NO: 57 (AZGP1), SEQ ID NO: 235 (ACSM3), and SEQ ID NO: 248 (CST4);
(c) SEQ ID NO: 1 (ADM), SEQ ID NO: 7 (C15orf48), SEQ ID NO: 11 (ETV4), SEQ ID NO: 49 (TDRD1), SEQ ID NO: 51 (TMC5), SEQ ID NO: 57 (AZGP1) and SEQ ID NO: 254 (GPR116); or
(d) SEQ ID NO: 8 (CRISP3), SEQ ID NO: 12 (F5), SEQ ID NO: 22 (HPN), SEQ ID NO: 35 (PEX10), SEQ ID NO: 39 (PLA2G7), SEQ ID NO: 44 (SLC10A7), SEQ ID NO: 59 (CSRP1), SEQ ID NO: 248 (CST4), SEQ ID NO: 254 (GPR116), SEQ ID NO: 261 (PCA3), and SEQ ID NO: 286 (SLC22A17).

5. The method of claim 1, wherein the plurality of RNA biomarkers consists of RNA biomarkers corresponding to the DNA sequences of:
(a) SEQ ID NO: 41 (PSMA), SEQ ID NO: 49 (TDRD1), SEQ ID NO: 241 (Clorf64), SEQ ID NO: 248 (CST4) and SEQ ID NO: 261 (PCA3);
(b) SEQ ID NO: 1 (ADM), SEQ ID NO: 7 (C15orf48), SEQ ID NO: 25 (KLK3), SEQ ID NO: 39 (PLA2G7), SEQ ID NO: 44 (SLC10A7), SEQ ID NO: 51 (TMC5), SEQ ID NO: 57 (AZGP1), SEQ ID NO: 235 (ACSM3) and SEQ ID NO: 248 (CST4);
(c) SEQ ID NO: 1 (ADM), SEQ ID NO: 7 (C15orf48), SEQ ID NO: 11 (ETV4), SEQ ID NO: 49 (TDRD1), SEQ ID NO: 51 (TMC5), SEQ ID NO: 57 (AZGP1) and SEQ ID NO: 254 (GPR116); or
(d) SEQ ID NO: 8 (CRISP3), SEQ ID NO: 12 (F5), SEQ ID NO: 22 (HPN), SEQ ID NO: 35 (PEX10), SEQ ID NO: 39 (PLA2G7), SEQ ID NO: 44 (SLC10A7), SEQ ID NO: 59 (CSRP1), SEQ ID NO: 248 (CST4), SEQ ID NO: 254 (GPR116), SEQ ID NO: 261 (PCA3) and SEQ ID NO: 286 (SLC22A17).

6. The method of claim 1, wherein the predetermined reference standard is established by measuring the expression level of the RNA biomarker in a plurality of biological samples selected from the group consisting of: (a) adjacent prostate gland samples obtained from the test subject; (b) prostate gland samples obtained from different, healthy, subjects; (c) a samples of prostatectomy gland tissue from prostatectomy samples that do not show primary tumors upon histological examination; (d) adjacent prostate gland samples obtained from different subjects with the same Gleason scores as the test subject; (e) adjacent prostate gland samples obtained from different subjects with different Gleason scores from the test subject; and (f) samples of normal human epithelial cells.

7. The method of claim 1, wherein the biological sample is selected from the group consisting of: urine, blood, serum, cell lines, peripheral blood mononuclear cells, biopsy tissue, and prostatectomy tissue.

8. The method of claim 1, wherein the expression levels of the plurality of RNA biomarkers is measured using next generation sequencing of an amplicon cDNA library prepared using a plurality of oligonucleotide primers specific for the plurality of RNA biomarkers.

9. The method of claim 8, wherein the plurality of oligonucleotide primers is selected from the group consisting of: SEQ ID NO: 76-232, 293-326 and 352-417.

10. A method for generating a prostate cancer differential expression profile for a subject, comprising:
(a) measuring expression levels of a plurality of RNA biomarkers in a biological sample obtained from the subject, wherein the plurality of RNA biomarkers comprises at least three RNA biomarkers selected from the group consisting of: RNA sequences corresponding to DNA sequences provided in SEQ ID NO: 1-75, 235-292, 327-351, 418 and 419;
(b) determining whether expression of each of the plurality of RNA biomarkers in the biological sample is up-regulated or down-regulated relative to a predetermined reference standard for each of the plurality of RNA biomarkers; and
(c) generating a prostate cancer differential expression profile for the test subject.

11. The method of claim 10, wherein the prostate cancer differential expression profile is generated in a format selected from the group consisting of: a database, an electronic display, a paper report, a text document, a graphic display, and a digital format.

12. The method of claim 10, wherein the at least three RNA biomarkers correspond to DNA sequences selected from the groups consisting of:
(a) SEQ ID NO: 41 (PSMA), SEQ ID NO: 49 (TDRD1), SEQ ID NO: 241 (C1orf64), SEQ ID NO: 248 (CST4) and 261 (PCA3);
(b) SEQ ID NO: 1 (ADM), SEQ ID NO: 7 (C15orf48), SEQ ID NO: 25 (KLK3), SEQ ID NO: 39 (PLA2G7), SEQ ID NO: 44 (SLC10A7), SEQ ID NO: 51 (TMC5), SEQ ID NO: 57 (AZGP1), SEQ ID NO: 235 (ACSM3) and SEQ ID NO: 248 (CST4);
(c) SEQ ID NO: 1 (ADM), SEQ ID NO: 7 (C15orf48), SEQ ID NO: 11 (ETV4), SEQ ID NO: 49 (TDRD1), SEQ ID NO: 51 (TMC5), SEQ ID NO: 57 (AZGP1) and SEQ ID NO: 254 (GPR116); or
(d) SEQ ID NO: 8 (CRISP3), SEQ ID NO: 12 (F5), SEQ ID NO: 22 (HPN), SEQ ID NO: 35 (PEX10), SEQ ID NO: 39 (PLA2G7), SEQ ID NO: 44 (SLC10A7), SEQ ID NO: 59 (CSRP1), SEQ ID NO: 248 (CST4), SEQ ID NO: 254 (GPR116), SEQ ID NO: 261 (PCA3) and SEQ ID NO: 286 (SLC22A17).

13. The method of claim 10, wherein the predetermined reference standard is established by measuring the expression level of the RNA biomarker in a plurality of biological samples selected from the group consisting of: (a) adjacent prostate gland samples obtained from the test subject; (b) prostate gland samples obtained from different, healthy, subjects; (c) samples of prostatectomy gland tissue from prostatectomy samples that do not show primary tumors upon histological examination; (d) adjacent prostate gland samples obtained from a plurality of different subjects with the same Gleason scores as the test subject; (e) adjacent prostate gland samples obtained from a plurality of different subjects with different Gleason scores from the test subject; and (f) samples of normal human epithelial cells.

14. The method of claim 10 wherein the biological sample is selected from the group consisting of: urine, blood, serum, cell lines, peripheral blood mononuclear cells, biopsy tissue, and prostatectomy tissue.

15. The method of claim 10, wherein the expression levels of the plurality of RNA biomarkers is measured using next generation sequencing of an amplicon cDNA library prepared using a plurality of oligonucleotide primers specific for the plurality of RNA biomarkers.

16. The method of claim 15, wherein the plurality of oligonucleotide primers is selected from the group consisting of: SEQ ID NO: 76-232, 293-326 and 352-417.

17. A method for establishing a reference standard for a RNA biomarker for use in diagnosing the presence of prostate cancer in a test subject comprising:
(a) measuring the expression level of the RNA biomarker in a plurality of biological samples selected from the group consisting of:
(i) prostate gland samples obtained from different, healthy, subjects;
(ii) samples of prostatectomy gland tissue from prostatectomy samples that do not show primary tumors upon histological examination;
(iii) adjacent prostate gland samples obtained from a plurality of different subjects with the same Gleason scores as the test subject; and
(iv) adjacent prostate gland samples obtained from a plurality of different subjects with different Gleason scores from the test subject; and
(b) determining the mean and the standard deviation of the expression level in the plurality of biological samples; and
(c) determining a lower end of a normal range of expression of the RNA biomarker as the mean minus two standard deviations, and determining an upper end of a normal range of expression of the RNA biomarker as the mean plus two standard deviations, thereby establishing the reference standard.

18. A method for determining whether a biomarker is differentially expressed in a prostate tissue sample obtained from a test subject, comprising:
(a) establishing a reference standard for the biomarker using the method of claim 20; and
(b) measuring the expression level of the biomarker in the prostate tissue sample obtained from the first subject; and
(c) determining whether the expression level of the biomarker in the prostate tumor sample obtained from the test subject is at a level that is: (i) more than 2.5 log₂ fold above or below the mean of the expression level of the biomarker in the at least one biological sample, and/or (ii) outside the normal range of expression of the biomarker in the plurality of biological samples, thereby determining whether the biomarker is differentially expressed in the prostate tissue sample obtained from the test subject.

19. A method for predicting a likelihood of reoccurrence or metastasis of prostate cancer in a subject, comprising:
(a) measuring the expression levels of a plurality of RNA biomarkers in a biological sample obtained from the subject, wherein the plurality of RNA biomarkers comprises at least three RNA biomarkers selected from the group consisting of: RNA sequences corresponding to DNA sequences provided in SEQ ID NO: 1-75, 235-292, 327-351, 418 and 419;
(b) comparing the expression level of each of the plurality of RNA biomarkers in the biological sample with a predetermined reference standard for the RNA biomarker; and
(c) predicting the likelihood of reoccurrence or metastasis of prostate cancer in the subject based on a comparison of the expression level of each of the plurality of RNA biomarkers with the predetermined reference standard for the RNA biomarker.

20. The method of claim 19, wherein the at least three RNA biomarkers correspond to DNA sequences selected from the groups consisting of:
(a) SEQ ID NO: 41 (PSMA), SEQ ID NO: 49 (TDRD1), SEQ ID NO: 241 (Clorf64), SEQ ID NO: 248 (CST4) and 261 (PCA3);
(b) SEQ ID NO: 1 (ADM), SEQ ID NO: 7 (C15orf48), SEQ ID NO: 25 (KLK3), SEQ ID NO: 39 (PLA2G7), SEQ ID NO: 44 (SLC10A7), SEQ ID NO: 51 (TMC5), SEQ ID NO: 57 (AZGP1), SEQ ID NO: 235 (ACSM3) and SEQ ID NO: 248 (CST4);
(c) SEQ ID NO: 1 (ADM), SEQ ID NO: 7 (C15orf48), SEQ ID NO: 11 (ETV4), SEQ ID NO: 49 (TDRD1), SEQ ID NO: 51 (TMC5), SEQ ID NO: 57 (AZGP1) and SEQ ID NO: 254 (GPR116); or
(d) SEQ ID NO: 8 (CRISP3), SEQ ID NO: 12 (F5), SEQ ID NO: 22 (HPN), SEQ ID NO: 35 (PEX10), SEQ ID NO: 39 (PLA2G7), SEQ ID NO: 44 (SLC10A7), SEQ ID NO: 59 (CSRP1), SEQ ID NO: 248 (CST4), SEQ ID NO: 254 (GPR116), SEQ ID NO: 261 (PCA3) and SEQ ID NO: 286 (SLC22A17).
